(19) United States
(12) Patent Application Publication
(10) Pub. No.: US 2016/0213797 A1
(43) Pub. Date: Jul. 28, 2016

(54) LOW ENERGY ELECTRON STERILIZATION

(71) Applicant: Electronworks Holdings LLC, Chicago, IL (US)

(72) Inventors: Daniel F. Gorzen, Ypsilanti, MI (US); Randol E. Kirk, Albuquerque, NM (US)

(21) Appl. No.: 15/091,039

(22) Filed: Apr. 5, 2016

Related U.S. Application Data
(63) Continuation of application No. 14/394,151, filed on Oct. 13, 2014, now abandoned.
(60) Provisional application No. 61/623,129, filed on Apr. 12, 2012.

(51) Int. Cl. A61L 2/08

(52) U.S. Cl. CPC A61L 2/087

(57) ABSTRACT

Low energy electron sterilizers and method of sterilization using low energy electrons are disclosed herein. An example method of sterilizing an instrument using low energy electrons can include generating one or more low energy electrons, maintaining the instrument in a vacuum and irradiating the instrument with the low energy electrons. The low energy electrons can have an energy less than or equal to 25 ke V, and the vacuum can be sufficiently low to prevent the one or more electrons from producing a plasma.

### CROSS-REFERENCE TO RELATED APPLICATIONS

This application claims the benefit of U.S. Provisional Patent Application No. 61/623,129, filed on Apr. 12, 2012, entitled “Systems and Methods for Sterilizing Items Using Low Energy Electrons,” the disclosure of which is expressly incorporated herein by reference in its entirety.

### I. BACKGROUND

We disclose systems and methods for reducing the bioburden, such as microbial, viral, and spores, on an organic or non-organic item. Some accepted methods for reducing the bioburden are irradiating an item with gamma or x-rays or bombarding the item with high energy electrons. Devices that deliver such beams typically emit high-energy beams, which are inappropriate for treating organic items. Using a high-energy beam on an organic item can lead to crosslinking, due to the production of radiolytics, molecular degradation, or combinations of these effects, as well as cause heat and molecular changes. The new systems and methods disclosed in this application mitigate or completely avoid those effects while still effectively reducing the bioburden to acceptable levels.

The disclosed systems and methods efficiently treat a wide variety of items using low-energy electrons. The systems and methods are disclosed in a variety of configurations adapted specifically for different types of items. The configurations are intended to be modular in some instances so that one system may be used for a variety of items and for multiple uses.

Previous devices have used electrons to sterilize items. Such devices typically generate and accelerate electrons to very high potentials measured in MeV in a vacuum and then pass the electrons through a window of very thin material before the electrons hit the target item. After passing through this window, the electrons travel through the air and lose energy before striking the target item. However, electrons still having high energies ionize the target item and kill any microbes in or on the product. By contrast, the disclosed systems and methods instead use very low energy electrons to reduce the microbial burden while reducing the production of x-rays and radiolytic compounds.

One objective of the disclosed systems and methods is to reduce the bioburden in the target item without materially affecting the properties of the target item.

### II. SUMMARY

Disclosed are systems and methods for reducing bioburden using electrons of relatively low energy. While knowledge has been developed for the interaction of low energy electrons and matter its applications have been almost solely in research. To develop a practical, usable irradiation system for reducing bioburden, a number of systems have to be controlled to successfully irradiate a product. Electrons can lose all ability to reduce bioburden unless the composition and pressure of the atmosphere in which the electrons are generated and through which the electrons flow is controlled. The degree and type of atmospheric control depends largely on the energy of the electrons. For low energy electrons, an atmosphere of air at a pressure of one bar is not feasible because such an atmosphere scatters the electrons and robs the electrons of the energy needed to kill microbial bioburden. Thus, when electrons are directed through too much atmosphere, the electrons lose their energy and become ineffective. However, low pressure atmospheres can create problems for some types of target items. Living tissue typically cannot be exposed to extremely low atmospheric pressures without causing damage.

Disclosed are devices and methods to provide a universal electron control module that allows any number of irradiation configurations to be used simply by attaching them to the electron control module. These configurations can contain different atmospheres, different beam paths, different sizes or different levels of acceleration and also include any combination of parts and components. The electrons used and disclosed herein can be generated by any mechanism, including an electron emitter, such as a filament, a cold cathode, or a dispenser cathode, photocathode, thermionic emitter, electron multipliers, carbon nanotubes, plasma source, broad area electron emitter, such as a planar emitter or linear emitter, piezoelectric electron emitter, among other devices, such as beta emitters. The electrons are generated inside an electron chamber. The target item is located in a product chamber that is positioned to permit the electrons to enter the chamber and strike the target item. The shape of the product chamber and/or the electron chamber can be any shape, including an ovoid shape, such as that of an egg. It is understood that in certain embodiments, the electron chamber and the product chamber can be effectively one in that they share the same atmosphere. In other embodiments, the electron and product chambers can be separated such that the atmospheres in each chamber are isolated from each other and can be controlled separately. In addition, the two chambers can become effectively a single chamber through an aperture or window which can be moved or opened or closed to either separate the atmospheres of the two chambers or mix the two atmospheres into one atmosphere. In embodiments, the atmospheres whether one atmosphere between the product and electron chambers or just the product chamber atmosphere, the atmosphere can be adjusted through vacuum, or in certain embodiments, and/or through addition of or replacement with an inert gas, such as helium. For the embodiments in which the product chamber is atmospherically distinct from the electron chamber, the atmosphere in the product chamber may be adjusted to accommodate a wide variety of target items, even including living tissue.

One exemplary embodiment of the sterilizer includes a product chamber and electron chamber having a single atmosphere which can be reduced to less than or equal to 25 milliTorr and an electron emitter capable of producing electrons of 1-40 keV. The electrons at this reduced pressure can be utilized for sterilization even though they are at energies of 1-40 kV. The effective impact energy of the electrons under these conditions is sufficient to reduce the bioburden without materially altering the original properties of the target item.

Low energy electron sterilizers are disclosed herein. An example sterilizer can include a low energy electron emitter and a product chamber. The low energy electron emitter can be configured to emit one or more electrons having an energy less than or equal to 25 keV into the product chamber.

Optionally, the product chamber can be sized to house at least an instrument. The instrument can optionally be a medical instrument. Additionally, the medical instrument can optionally require an amount of sterilization for use in a medical environment. For example, the amount of sterilization can be a 2, 3, 4, 5, or 6 log reduction in bioburden. Example medical instruments include at least one of a syringe, a scissor and a scalpel.

Alternatively or additionally, the instrument can optionally have at least one dimension of approximately 10-16 inches or greater. Alternatively, the instrument can optionally have at least one dimension between approximately 5-10 inches. Alternatively, the instrument can optionally have at least one dimension between approximately 3-5 inches.

Additionally, the product chamber can optionally have a volume of at least one of approximately 0.5 L, 0.8, 1.0, 1.5, 2.0, 2.5, 3.0, 5.0, 10.0, 20, 30, 50, or 100 L. Alternatively or additionally, the product chamber can have at least one dimension of 0.1, 0.3, 0.5, 0.7, 1.0, 1.5, 2.0, 3.0, 5.0, or 10.0 meters. Optionally, the product chamber can have a rectangular, square, spherical, or ovoid shape.

Additionally, the product chamber can be maintained at a vacuum sufficiently low to prevent the one or more electrons from producing a plasma. For example, the product chamber can be maintained at a vacuum less than or equal to 10⁻² Torr. Alternatively or additionally, the product chamber can be maintained at a vacuum less than or equal to 10⁻³ Torr.

The energy electron emitter and the product chamber can optionally be under a same atmosphere. For example, the sterilizer can further include an electron chamber configured to house the low energy electron emitter. The electron chamber and the product chamber can optionally be in fluid communication. For example, an aperture can be arranged between the electron chamber and the product chamber. The aperture can be at least one of a hole, a mesh, a gate valve or a thin foil.

The sterilizer can further include a vacuum pump that is operatively connected to the product chamber. The vacuum pump can be configured to generate and maintain the vacuum. Alternatively or additionally, the sterilizer can further include a product support apparatus contained in the product chamber. The product support apparatus can optionally be configured to maintain a target of sterilization stationary. Optionally, the sterilizer can include an agitator that is operably connected to the product support apparatus. The agitator can be configured to move the product support apparatus. Alternatively or additionally, the sterilizer can include a high voltage generator that is operably coupled to the low energy electron emitter. For example, the high voltage generator can be configured to generate at least a voltage of between 5 kV and 25 kV.

Optionally, the low energy electron emitter can be at least one of a filament, a cold cathode, or a dispenser cathode, an oxide coated cathode, a photocathode, a thermionic emitter, an electron multiplier and a plasma. Alternatively or additionally, at least a portion of the product chamber can be coated with a high Z number material. Additionally, at least one of the electrons can be backscattered at least one time in the product chamber. Optionally, at least one of the electrons can be backscattered at least 2, 3, 4, 5, 6, or 7 times.

Another example sterilizer can include a low energy electron emitter and a product chamber sized to house at least an instrument. The low energy electron emitter can be configured to emit one or more electrons into the product chamber, and the product chamber can be maintainable at a vacuum less than or equal to 10⁻² Torr. Optionally, the product chamber can be maintainable at a vacuum less than or equal to 10⁻³ Torr.

Optionally, the product chamber can be sized to house at least an instrument. The instrument can optionally be a medical instrument. Additionally, the medical instrument can optionally require an amount of sterilization for use in a medical environment. For example, the amount of sterilization can be a 2, 3, 4, 5, or 6 log reduction in bioburden. Example medical instruments include at least one of a syringe, a scissor and a scalpel.

Alternatively or additionally, the instrument can optionally have at least one dimension of approximately 10-16 inches or greater. Alternatively, the instrument can optionally have at least one dimension between approximately 5-10 inches. Alternatively, the instrument can optionally have at least one dimension between approximately 3-5 inches.

Additionally, the product chamber can optionally have a volume of at least one of approximately 0.5 L, 0.8, 1.0, 1.5, 2.0, 2.5, 3.0, 5.0, 10.0, 20, 30, 50, or 100 L. Alternatively or additionally, the product chamber can have at least one dimension of 0.1, 0.3, 0.5, 0.7, 1.0, 1.5, 2.0, 3.0, 5.0, or 10.0 meters. Optionally, the product chamber can have a rectangular, square, spherical, or ovoid shape.

Optionally, the low energy electron emitter can be configured to emit one or more electrons having an energy sufficiently low to prevent the one or more electrons from producing a plasma. Alternatively or additionally, the low energy electron emitter can be configured to emit one or more electrons having an energy less than or equal to 100 keV. Alternatively, the low energy electron emitter can be configured to emit one or more electrons having an energy in a range between 10-50 keV.

The energy electron emitter and the product chamber can optionally be under a same atmosphere. For example, the sterilizer can further include an electron chamber configured to house the low energy electron emitter. The electron chamber and the product chamber can optionally be in fluid communication. For example, an aperture can be arranged between the electron chamber and the product chamber. The aperture can be at least one of a hole, a mesh, a gate valve or a thin foil.

The sterilizer can further include a vacuum pump that is operatively connected to the product chamber. The vacuum pump can be configured to generate and maintain the vacuum. Alternatively or additionally, the sterilizer can further include a product support apparatus contained in the product chamber. The product support apparatus can optionally be configured to maintain a target of sterilization stationary. Optionally, the sterilizer can include an agitator that is operably connected to the product support apparatus. The agitator can be configured to move the product support apparatus. Alternatively or additionally, the sterilizer can include a high voltage generator that is operably coupled to the low energy electron emitter. For example, the high voltage generator can be configured to generate at least a voltage of at least 100 kV. Alternatively, the high voltage generator can be configured to generate at least a voltage of at least 10 kV.

Optionally, the low energy electron emitter can be at least one of a filament, a cold cathode, or a dispenser cathode, an oxide coated cathode, a photocathode, a thermionic emitter, an electron multiplier and a plasma. Alternatively or additionally, at least a portion of the product chamber can be coated with a high Z number material. Additionally, at least one of the electrons can be backscattered at least one time in the product chamber. Optionally, at least one of the electrons can be backscattered at least 2, 3, 4, 5, 6, or 7 times.

Another example sterilizer can include an electron chamber for housing a low energy electron emitter, a product chamber sized to house a instrument and a window arranged between the electron chamber and the product chamber. The low energy electron emitter can be configured to emit one or more electrons having an energy less than or equal to 25 keV into the product chamber through the window. Additionally, the electron chamber and the product chamber can be maintainable at a vacuum less than or equal to 10⁻² Torr. Optionally, the product chamber can be maintainable at a vacuum less than or equal to 10⁻³ Torr.

Optionally, the product chamber can be sized to house at least an instrument. The instrument can optionally be a medical instrument. Additionally, the medical instrument can optionally require an amount of sterilization for use in a medical environment. For example, the amount of sterilization can be a 2, 3, 4, 5, or 6 log reduction in bioburden. Example medical instruments include at least one of a syringe, a scissor and a scalpel.

Alternatively or additionally, the instrument can optionally have at least one dimension of approximately 10-16 inches or greater. Alternatively, the instrument can optionally have at least one dimension between approximately 5-10 inches. Alternatively, the instrument can optionally have at least one dimension between approximately 3-5 inches.

Additionally, the product chamber can optionally have a volume of at least one of approximately 0.5 L, 0.8, 1.0, 1.5, 2.0, 2.5, 3.0, 5.0, 10.0, 20, 30, 50, or 100 L. Alternatively or additionally, the product chamber can have at least one dimension of 0.1, 0.3, 0.5, 0.7, 1.0, 1.5, 2.0, 3.0, 5.0, or 10.0 meters. Optionally, the product chamber can have a rectangular, square, spherical, or ovoid shape.

The sterilizer can further include a vacuum pump that is operatively connected to the product chamber. The vacuum pump can be configured to generate and maintain the vacuum. Alternatively or additionally, the sterilizer can further include a product support apparatus contained in the product chamber. The product support apparatus can optionally be configured to maintain a target of sterilization stationary. Optionally, the sterilizer can include an agitator that is operably connected to the product support apparatus. The agitator can be configured to move the product support apparatus. Alternatively or additionally, the sterilizer can include a high voltage generator that is operably coupled to the low energy electron emitter. For example, the high voltage generator can be configured to generate at least a voltage of between 5 kV and 25 kV.

Optionally, the low energy electron emitter can be at least one of a filament, a cold cathode, or a dispenser cathode, an oxide coated cathode, a photocathode, a thennionic emitter, an electron multiplier and a plasma. Alternatively or additionally, at least a portion of the product chamber can be coated with a high Z number material. Additionally, at least one of the electrons can be backscattered at least one time in the product chamber. Optionally, at least one of the electrons can be backscattered at least 2, 3, 4, 5, 6, or 7 times.

Also disclosed herein are methods for sterilization using low energy electrons. An example method of sterilizing an instrument using low energy electrons can include generating one or more low energy electrons, maintaining the instrument in a vacuum and irradiating the instrument with the low energy electrons. The low energy electrons can have an energy less than or equal to 25 keV, and the vacuum can be sufficiently low to prevent the one or more electrons from producing a plasma. Optionally, the vacuum can be less than or equal to 10⁻² Torr. Optionally, the instrument can be sterilized to achieve a 2, 3, 4, 5, or 6 log reduction in bioburden. Optionally, the instrument can be a medical instrument.

Another example method of sterilizing an instrument using a low energy electron sterilizer can include generating one or more low energy electrons, guiding the one or more low energy electrons into a product chamber of the sterilizer and irradiating the instrument with the low energy electrons. The low energy electrons can have an energy less than or equal to 25 keV. Optionally, the product chamber can be maintained at a vacuum sufficiently low to prevent the one or more low energy electrons from producing a plasma.

Yet another example method of sterilizing an instrument using a low energy electron sterilizer can include generating one or more low energy electrons, guiding the one or more low energy electrons into a product chamber of the sterilizer and irradiating the instrument with the low energy electrons. The product chamber can be maintainable at a vacuum sufficiently low to prevent the one or more low energy electrons from producing a plasma. Optionally, the low energy electrons can have an energy sufficiently low to prevent the low energy electrons from producing a plasma. Alternatively or additionally, the low energy electrons can have an energy less than or equal to 100 keV. Alternatively, the low energy electron emitter can be configured to emit one or more electrons having an energy in a range between 10-50 keV.

Optionally, it should be understood that the method of sterilization can be implemented using any of the sterilizers discussed herein.

Another example sterilizer can include a sterilization chamber, an atmosphere inside the sterilization chamber, an electron emitter inside the sterilization chamber that is configured to emit electrons, a target holder inside the sterilization chamber and a pump. The pump can be operatively connected to the sterilization chamber and capable of altering the atmosphere inside the sterilization chamber. The electron emitter and the target holder are at all times both exposed to the atmosphere.

Optionally, the atmosphere is at a pressure of no more than 50 milliTorr. Alternatively, the atmosphere includes mostly an inert gas. For example, the inert gas can be helium.

Additionally, the pump can be adapted for reducing the pressure of the atmosphere to less than 50 milliTorr. Alternatively or additionally, the pump is adapted for exchanging a first gas in the atmosphere for a second gas in the atmosphere. Additionally, the second gas is substantially an inert gas. For example, the inert gas can be substantially nitrogen. Alternatively, the inert gas can be substantially helium.

Alternatively or additionally, the sterilization chamber further includes an electron chamber and a product chamber in fluid communication with the electron chamber. Optionally, the electron chamber further includes a first connector and the product chamber further includes a second connector that is adapted to mate with the first connector. Optionally, an aperture is provided in the electron chamber capable of allowing transmission of at least the electrons. For example, the aperture can be selected from a group consisting of a pin hole, mesh, gate valve, or thin foil.

Optionally, the sterilizer can further include a high voltage generator operably linked to the electron emitter. The high voltage generator can generate voltages of between about 5 kV and 50 kV. Optionally, the voltages are between 10 kV and 40 kV. Optionally, the voltages are between 10 kV and 25 kV. Optionally, the voltages are between 5 kV and 15 kV.

Additionally, another example sterilizer can include an electron chamber containing a first atmosphere, an electron emitter inside the electron chamber that is configured to emit electrons, a product chamber containing a second atmosphere that does not mix with the first atmosphere, a target holder inside the product chamber, and a pump that is operatively connected to the electron chamber and capable of altering the pressure and composition of the first atmosphere.

Optionally, the pump is operatively connected to the product chamber and capable of altering the pressure or composition of the second atmosphere.

Alternatively or additionally, the electron emitter is a filament, a cold cathode, or a dispenser cathode, an oxide coated cathode, a photocathode, a thermionic emitter, an electron multiplier or a plasma.

Optionally, the sterilization chamber and/or the product chamber is an ellipsoid. Alternatively, the sterilization chamber and/or the product chamber is a sphere.

Optionally, the sterilization chamber includes an agitator connected to the target holder. The agitator can vibrate the target holder. Alternatively or additionally, the agitator can rotate the target holder.

Optionally, a coating is provided on at least a portion of the electron chamber having a z-rating of at least about 3.5. Alternatively or additionally, the product chamber is glass, stainless steel, ceramic, plastic, or high Z-material.

Optionally, the product chamber includes a high Z-material has an atomic number of at least 50.

Alternatively or additionally, the sterilizer further includes a first valve to control the flow of gases into or out of the electron chamber. The sterilizer can also optionally include a second valve to control the flow of gases into or out of the product chamber. Alternatively or additionally, the sterilizer can include a first valve to control the flow of gases into or out of the electron chamber and the product chamber.

Optionally, the sterilizer includes a focusing ring for influencing the direction or shape or both of the electrons.

Additionally, the impact energy of electrons can optionally be less than or equal to 50 kV, 40 kV, 30 kV, 25 kV, 15 kV, 10 kV, or 5 kV. Alternatively or additionally, the product chamber, the electron chamber and/or the sterilization chamber can withstand at least a 5 millitor, 10 millitor, 50 millitor, 100 millitor, 1 millitor, 0.5 millitor, 0.3 millitor, 0.1 millitor, 0.05 millitor, 0.01 millitor, 0.005 millitor, 0.001 millitor, 0.0005 millitor, 0.0001 millitor, 0.00005 millitor, 0.00001 millitor, 0.000005 millitor, or 0.000001 millitor vacuum.

An example irradiator can include a sterilization chamber, where the sterilization chamber includes an electron emitter and a product support apparatus. The sterilization chamber can also include a vacuum pump that is operably linked to the sterilization chamber, such that when the vacuum pump produces a vacuum, the electron emitter and the product support apparatus are under the same atmosphere.

Additionally, the sterilization chamber can optionally include an electron chamber and product chamber operably linked via an aperture. The operable linkage can be a connector. For example, the connector can be a male-female connector. The atmosphere of the electron chamber and the atmosphere of the product chamber can be in free exchange after the aperture is opened.

The irradiator can optionally include a gas exchanger. Alternatively or additionally, the irradiator can optionally include a manifold. The manifold can be operably connected to the product chamber. Alternatively or additionally, the manifold can be operably connected to a vacuum pump and a gas exchanger. The gas exchanger can exchange helium, hydrogen, residual atmosphere, and water vapor. In addition, the irradiator can include a second manifold operably connected to the electron chamber, operably connected to a second vacuum source. Optionally, the irradiator can include valves to independently control the vacuum and gas in each chamber.

Additionally, the irradiator can include a high voltage generator operably connected to the electron emitter. The generator can provide a delivered energy of 4 kV to 30 kV. Optionally, the delivered energy is 7 kV to 20 kV. Optionally, the delivered energy is 9 kv to 13 kV. Optionally, the delivered energy is 10 kV.

The irradiator can optionally include a focusing ring. Alternatively or additionally, the irradiator can include a gate valve. Alternatively or additionally, the irradiator can include a valve for reducing the pressure of the product chamber.

Optionally, the irradiator can include a pressure transducer. The pressure transducer can be a vacuum indicator.

Alternatively or additionally, the irradiator can include a vibrator, and the vibrator can connect to the product support apparatus such that the product support apparatus can be vibrated.

Optionally, the irradiator can include an electron multiplier.

Alternatively or additionally, the irradiator can contain a modified atmosphere to allow electron travel with a calculated electron energy reduction per unit length through the chamber to the product.

The radiator can optionally include a rotating drum for holding the product. Alternatively or additionally, the product chamber can include a cylindrical container in vertical axis alignment with the emission of electrons.

Optionally, the irradiator can include a laser device.

Optionally, the irradiator can include a wire mesh net for suspension of an endoscope in extended alignment along a rod. Alternatively or additionally, the irradiator can include at least one electromagnet. Optionally, the product support apparatus can be a mesh tray or a mesh bag. For example, the mesh bag can be formed from a conductive material.

Additionally, the irradiator can include a heat (pressure) sensitive holder, facing toward the electron source.

Alternatively or additionally, the generator provides a delivered energy of 4 kV to 30 kV. Optionally, the delivered energy is 7 kV to 20 kV. Optionally, the delivered energy is 9 kv to 13 kV. Optionally, the delivered energy is 10 kV.

Optionally, a method of sterilizing an instrument can be implemented using any one of the sterilizers and/or irradiators disclosed herein.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 schematically illustrates one embodiment of a sterilizer having an electron chamber that can be atmospherically isolated from a product chamber. Shown is a cabinet housing (1), with an electron chamber (2), a product chamber (3), a vacuum gas manifold (4), power supply (producing voltages of for example, 10, 20, 30, 40, or 50 kV or more) (5), a vacuum pump (6), an inert gas cylinder (7), a vacuum pipe (8), a high voltage cable (9), and a valve (10).

FIG. 2 schematically illustrates a product chamber of the type shown in FIG. 1. Shown are a product chamber (3), a valve (10), a coupling connector, for example, a female connector (F) as appropriate (17), adjustable atmosphere (18), a concave circular container (19), an agitator (20), and a rotational motor and shaft (21).

FIG. 3 schematically illustrates elements the electron chamber shown in FIG. 1. Shown are an electron chamber (2), a high voltage cable (9), a valve (10), a filament (11), an aperture (12), a coupling connector, for example, a male connector (M) as appropriate (13), electrons (14), an adjusted atmosphere in chamber (15), and a focusing cup (16).

FIG. 4 schematically illustrates a mesh tray for holding the target item inside a product chamber 3 such as the one shown in FIG. 1. Shown are an electron chamber (2), a product chamber (3), an adjusted atmosphere (18), a coupling connector F (17), a coupling connector M (13), a mesh platform (30), and a vibrating coil (31).

FIG. 5 schematically illustrates an alternative embodiment of a product chamber in which the target item is held in a mesh bag and a system for encasing the sterilized target item in a film while the item is still inside the product chamber in order to preserve the sterile condition of the target item. Shown are an electron chamber (2), a product chamber (3), a coupling connector F (17), a coupling connector M (13), an access door (32), a heat sealer top (33), a heat sealer bottom (34), a top container cover (35), a bottom container cover (36), an entrance rollers top (37), an entrance rollers bottom (38), a rotation motor (39), and a mesh bag (40).

FIG. 6 schematically illustrates an alternative embodiment of a sterilizer suitable for sterilizing living tissue. Shown are an electron chamber (2), a product chamber (Gas shroud) (3), a valve (10), a coupling connector F (17), a coupling connector M (13), a helium positive flow (41), a convex (electron permeable window) (42), an x-y travel for controlled motion (43), and trapped gas (44).

FIG. 7 schematically illustrates another alternative embodiment suitable for sterilizing living tissue, with a glovebox type region with trapped gas in it, described as a flexible non-porous drape. Shown are a body product cover, an electron chamber (2), a valve (10), a convex (electron permeable window) (42), and trapped gas (44), a flexible non-porous drape (45), a non-permiablizable drape (46).

FIG. 8 schematically illustrates the major components of an embodiment of a sterilizer.

FIG. 9 shows the results of a sterilization activity.

FIGS. 10A and 10B depict the probability results of an electron scatter.

FIG. 11 schematically illustrates an electron emitter suited for use in the sterilizer. Shown is a section view, an emitter (100), a −15 kV power supply (105), a high voltage cable (120), a high voltage receptacle (130), a vacuum enclosure (140), a focusing cup (150), electrons (300), a turbo pump (170), a rough pump (180), a pressure gauge (190), a product in a rotating bowl (200), deflection focusing coils (160), iridium filament coated with Yttria (110).

FIG. 12 illustrates a plasma electron source suitable for use in the sterilizer.

FIG. 13 illustrates a plasma electron source suitable for use in the sterilizer while in operation.

### IV. DETAILED DESCRIPTION

A typical requirement of hospital operating rooms and surgery centers is the ability to rapidly sterilize medical instruments for reuse. However, in most situations, the current technology fails to provide a workable solution. For example, specifically, a need exists to be able to recycle specialty instruments or implant parts that may have moved outside the sterile field during short time available during a surgical procedure. These requirements can be particularly important in developing, rural, and mobile healthcare settings where budgets for equipment are lower and facilities are more primitive. Current sterilizers such as autoclaves require a long, such as 15-20 or 30 plus minute cycle from the time the instrument is placed in the sterilizer until it is cool enough to handle after sterilization. Autoclaves also require significant cost and infrastructure to function properly.

The disclosed systems and methods in certain embodiments can, for example, reduce the time needed to sterilize a surgical instrument or implant to four minutes or less, for example, sterilization in seconds to tens of seconds, or even less, with cycle times of tens of seconds to minutes, or less. The disclosed sterilizing systems use a relatively low voltage power supply attached to a vacuum chamber. A vacuum chamber can include an electron chamber, a product chamber or a combination of the electron and vacuum chambers as discussed in detail below. Alternatively or additionally, a sterilization chamber can include the electron chamber and/or the product chamber. Thus, a vacuum chamber can also be a sterilization chamber. Optionally, the electron chamber and the product chamber can share a same or different atmosphere as discussed in detail below. In the chamber or attached to the chamber is located an electron emitter (can be insensitive to moderate vacuum effects) and an acceleration grid to provide a stream of electrons within the chamber. A self-sealing door in the chamber allows a holder, such as a basket to be placed in the chamber containing an instrument to be sterilized. The chamber can be sealed and evacuated to, for example, 10⁻⁴ Torr. Using turbo molecular pump technology, such as in production line “vacuum leak detector” units, such a vacuum can be achieved in less than 1 minute, such as under 10 seconds. Optionally, the chamber can be sealed and evacuated to other levels of vacuum such as 10⁻² Torr, 10⁻³ Torr, 10⁻⁴ Torr, 10⁻⁵ Torr, 10⁻⁶ Torr, etc., for example. A lower vacuum as provided herein refers to a lower absolute pressure. For example, a vacuum of 10⁻⁴ Torr is considered lower than a vacuum of 10⁻³ Torr. After the chamber is evacuated, an electron generator emits a field of electrons and a series of electromagnetic coils selectively steer the beam and shape its cross section as it travels through the chamber. In certain embodiments, appropriate steering and shaping of the beam ensures that the entire surface of the targeted surgical instrument or implant is exposed to the sterilizing effect of the electrons. The beam can also be expanded or contracted to insure all shielded or shadowed surfaces are cleaned, or in certain embodiments the bouncing of the electrons can be sufficient.

The sterilizers, and in particular the product chambers and/or the sterilization chambers (discussed below), can be sized to house at least an instrument. The instrument can optionally be a medical instrument. Alternatively, this disclosure contemplates that the instrument can be an instrument other than a medical instrument and can include any instrument that requires an amount of sterilization such as a 2, 3, 4, 5, or 6 log reduction in bioburden, for example. The amount of sterilization can optionally be more or less than a 6 log reduction.

Alternatively or additionally, the instrument can optionally have at least one dimension of approximately 10-16 inches or greater. Alternatively, the instrument can optionally have at least one dimension between approximately 5-10 inches. Alternatively, the instrument can optionally have at least one dimension between approximately 3-5 inches.

Additionally, the product chamber can optionally have a volume of at least one of approximately 0.5 L, 0.8, 1.0, 1.5, 2.0, 2.5, 3.0, 5.0, 10.0, 20, 30, 50, or 100 L. Alternatively or additionally, the product chamber can have at least one dimension of 0.1, 0.3, 0.5, 0.7, 1.0, 1.5, 2.0, 3.0, 5.0, or 10.0 meters. Optionally, the product chamber can have a rectangular, square, spherical, or ovoid shape.

A non-inclusive list of medical instruments includes syringes, scissors, forceps, knives/blades, scalpels, catheters, needles, etc. Alternatively or additionally, medical instruments include articulators, clamps, fasteners (e.g., screws), tubing, diagnostic instruments, specula, chisels, box joints, needle holders, cannula, drill bits, retractors, saws, implantable devices, shears, etc. It should be understood that medical instruments can have a variety of sizes and a variety of shapes. Alternatively or additionally, medical instruments can include one or more parts including moving parts. Further, medical instruments can have one or more crevasses, cracks and/or recessed portions/areas due to their shapes, sizes and/or number of parts.

A non-limiting list of classes of medical instruments which can be sterilized in some or all of the embodiments of the disclosed sterilizers are Articulator, Bone chisel, Cottle cartilage crusher, Bone cutter, Bone distractor, Ilizarov apparatus, Intramedullary kinetic bone distractor, Bone drill, Bone extender, Bone file, Bone lever, Bone mallet, Bone rasp, Bone saw, Bone skid, Bone splint, Bone button, Caliper, Cannula, Catheter, Cautery, Clamps, Curette, Depressor, Dilator, Dissecting knife, Distractor, Dermatome, Forceps, Forceps, Dissecting, Forceps, Tissue, Forceps (Other), Acanthulus or Acanthabolos, Bone forceps, Carmalt forceps, Cushing forceps, Dandy forceps, DeBakey forceps, Doycn intestinal forceps, Epilation forceps, Halstead forceps, Kelly forceps, Kocher forceps, Mosquito forceps, Hemostat, Hook, Nerve hook, Obstetrical hook, Skin hook, Hypodermic needle, Lancet (scalpel), Luxator, Lythotome, Lythotript, Mallet, Partsch mallet, Mouth prop, Mouth gag, Mammotome, Needle holder, Occluder, Osteotome, Epker osteotome, Periosteal elevator, Joseph elevator, Molt periosteal elevator, Obweg periosteal elevator, Septum elevator, Tessier periosteal elevator, Probe, Retractor, Senn retractor, Gelpi retractor, Weitlaner retractor, USA-Army/Navy retractor, O'Connor-O'Sullivan, Deaver, Bookwalter, Sweetheart[disambiguation needed], Joseph Skin Hook, Lahey Retractor, Blair (Rollet) Retractor, Rigid Rake, Flexible Rake, Ragnell Retractor, Linde-Ragnell Retractor, Davis Retractor, Volkman Retractor, Mathieu Retractor, Jackson Tracheal Hook, Crile Retractor, Meyerding Finger Retractor, Little Retractor, Love Nerve Retractor, Green Retractor, Goelet Retractor, Cushing Vein Retractor, Langenbeck Retractor, Richardson Retractor, Richardson-Eastmann Retractor, Kelly Retractor, Parker Retractor, Parker-Mott Retractor, Roux Retractor, Mayo-Collins Retractor, Ribbon Retractor, Alm Retractor, Self Retaining Retractors, Weitlaner Retractor, Beckman-Weitlaner Retractor, Beckman-Eaton Retractor, Beckman Retractor, Adson Retractor, Rib spreader, Rongeur, Ultrasonic scalpel, Laser scalpel, Scissors, Iris scissors, Kiene scissors, Metzenbaum scissors, Mayo scissors, Tenotomy scissors, Spatula, Speculum, Mouth speculum, Rectal speculum, Sim's vaginal speculum, Cusco's vaginal speculum, Sponge bowl, Sterilization tray, Sternal saw, Suction tube, Surgical elevator, Surgical hook, Surgical knife, Surgical mesh, Surgical needle, Surgical snare, Surgical sponge, Surgical spoon, Surgical stapler, Surgical tray, Suture, Syringe, Tongue depressor, Tonsillotome, Tooth extractor, Towel clamp, Towel forceps, Backhaus towel forceps, Lorna towel forceps, Tracheotome, Tissue expander, Subcutaneous inflatable balloon expander, Trephine, Trocar, Tweezers, Venous clipping, Stethoscope, Reflex testing hammer (padded), Sphygmomanometer (Blood pressure meter), A thin beam electric torch, A watch/stopwatch, A measuring tape, A weighing machine, Tuning forks, Kidney dish, Bedpan, Thermometer, Gas cylinders, Oxygen mask or tubes, Vaporizer, Instrument sterilizers, Dressing drums, Nebulizer, Positive pressure ventilator, Cardioverter/Defibrillator, Dialyser, Rubber catheter, Syringe of different sizes and needles, Canula, Transfusion sets, Sucker, Gastrointestinal tubes, Nasogastric tube, Stomach tube, Levin's tube, Kehr's “T” tube, Infant feeding tube, Spectacles, Enema set, Bandage, Pipettes or droppers, Graduated spoons, Ophthalmoscope, Otoscope, Endoscope, Proctoscope, bottle stands, Gauze, cotton, antiseptics, and gloves.

Examples of specific instruments include Galotti articulator, Castroviejo caliper, Payr pylorus clamp, Adson, Allis Babcock, Sponge Forceps, kalabasa, Castroviejo Crilewood, Mayo-Hegar, Olsen-Hegar.

A certain set of artery forceps that can be sterilized in the disclosed sterilizers includes, Haemostatic Kelly forceps, box joint, straight/curved 5½″ (14 cm), Haemostatic Crile forceps, box joint, straight/curved 5½″ (14 cm), Haemostatic Crile baby forceps, box joint, straight/curved 5½″ (14 cm), Haemostatic hospital (Kilner) forceps, box joint 5½″ (14 cm), Haemostatic Rochester Pean forceps, box joints, straight/curved (5″ (13 cm), 5½″ (14 cm), 6¼″ (16 cm), 7″ (18 cm), 8″ (20 cm)), Haemostatic Rochester Ochsner forceps, box joint, straight/curved 6¼″ (16 cm), 7″ (18 cm), 8″ (20 cm), Haemostatic Cha forceps, box joint, straight/curved 5″ (13 cm), 5½″ (14 cm), 6″ (15 cm), Haemostatic Rochester Carmalt forceps, box joint, straight/curved 6¼″ (16 cm), ″ (18 cm), 8″ (20 cm), Haemostatic Rochester Carmalt-Ochsner forceps, box joint, straight/curved 6½″ (16 cm), 7″ (18 cm), 8″ (20 cm), Haemostatic Heaney forceps, box joint 8″ (20 cm), Haemostatic “Dudfield Rose” forceps, box joint 10¼″ (26 cm), Haemostatic Mixter forceps, box joint 6¼″ (16 cm), 7¼″ (18½ cm), 9″ (23 cm), Haemostatic Finochietto forceps, box joint 9½″ (24 cm), Haemostatic Wertheim-Cullen forceps, box joint (21½ cm), Haemostatic Wertheim forceps, box joint 9½″ (24 cm), Haemostatic Crafoord forceps jaws with horizontal serrations, box joint 9½″ (24 cm), Haemostatic Crafoord-Sellors forceps, jaws with longitudinal serrations, box joint 9½″ (24 cm), Haemostatic Collin's Sellors forceps, Collin lock, oval jaws 6¼″ (16 cm), 7″ (18 cm), Haemostatic Collin's Sellors forceps, Collin lock, heart shaped 6¼″ (16 cm), 7″ (18 cm), Haemostatic Collin's Sellors forceps, Collin lock, t-shaped 6¼″ (16 cm), 7″ (18 cm), Haemostatic Collin's Sellors forceps, Collin lock, rhomboidal 6¼″ (16 cm), 7″ (18 cm), Spencer Wells artery forceps, screw joint, straight/curved 5″ (13 cm), 6″ (15 cm), 7″ (18 cm), 8″ (20 cm), 9″ (23 cm), Spencer Wells artery forceps, box joint, straight/curved 5″ (13 cm), 6″ (15 cm), 7″ (18 cm), 8″ (20 cm), 9″ (23 cm), Peans artery forceps screw joint 5″ (13 cm), 5¼″ (14 cm), 6″ (15 cm), Dunhill's artery forceps, screw joint, curved on flat 5″ (13 cm), Halstead's Mosquito artery forceps, screw joint, straight/curved 5″ (13 cm), 5″ (13 cm), Box joint 5″ (13 cm), Halstead's Mosquito Kocher artery forceps, 1×2 teeth box joint, straight/curved 5″ (13 cm), and Kochers artery forceps, box joint, straight/curved 5″ (13 cm), 5½″ (14 cm), 6″ (15 cm), 7″ (18 cm), 8″ (20 cm).

A certain set of diagnostic instruments that can be sterilized in the disclosed sterilizers includes, BOWLES STETHOSCOPE with metal chest piece 45 or 50 mm, rubber tubing and metal binaural folding type, C/P. FORD BOWLES combination stethoscope with raised diaphragm, DEEP BELL and double outlet, rubber tubing, metal binaural folding type chrome plated, FORD STETHOSCOPE with chest piece, rubber tubing and metal binaural folding type chrome plated, STETHOSCOPE BINAURAL METAL, standard type with ear tips. C/P, STETHOSCOPE BINAURAL METAL, folding type with ear tips. C/P, FORD'S CHEST piece with black plastic deep bell chrome plated, FORD BOWLES combination chest piece raised diaphragm, black plastic deep bell, single outlet, chrome plated, BOWLES CHEST PIECE medium size 1¾″ (45 mm) with raised diaphragm double outlet, chrome plated, BOWLES CHEST PIECE medium size 1¾″ (45 mm) with raised diaphragm, single outlet, chrome plated, BOWLES CHEST PIECE medium size 1¾″ (45 mm) with raised diaphragm, single outlet, chrome plated, STETHOSCOPE, wooden, STETHOSCOPE PINARD, aluminum, TYLOR PERCUSSION HAMMER, loop handle, TYLOR PERCUSSION HAMMER, solid handle, DEJERINE PERCUSSION HAMMER, and DEJERINE PERCUSSION HAMMER

A certain set of eye instruments that can be sterilized in the disclosed sterilizers includes, Troeltsch's ear specula, adult size, set of 3, Troeltsch's ear specula, infant size, set of 3, Toynbee ear specula, adult size, set of 3, Hartmann ear specula, adult size, set of 3, Gruber ear specula, adult size, set of 4, Erhardt ear specula, adult size, set of 3, Hartmann eustachian catheter, Troestsch eustachian catheter, Krause ear snares 7″ (17.5 cm), Hartmann's ear dressing forceps, very delicate pattern 5¼″ (14 cm), Hartmann's ear dressing forceps, standard pattern 6″ (15 cm), Troelrsch's ear dressing forceps, serrated 5″ (13 cm), Troelrsch's ear dressing forceps, 1×2 teeth, 5″ (13 cm), Troeltsch's ear dressing forceps, serrated, cross action 5¼″ (14 cm), Lucae's ear dressing forceps, serrated, cross action 5¼″ (14 cm), Lucae's ear dressing forceps, 1×2 teeth 5½″ (14 cm), Lucae's compression forceps, Ear syringe, 2 oz, 1 shield, 3 nozzel, Ear syringe, 3 oz, 1 shield, 3 nozzel, Ear syringe, 4 oz, 1 shield, 3 nozzel, Ear syringe, 6 oz, 1 shield, 3 nozzel, Ear syringe, 8 oz, 1 shield, 3 nozzel, Ear polypus forceps, crocodile action, Buckley mastoied chisels, Trautmann mastoied chisels, 2, 4, 6, 8, 10 mm, Schwartze mastoied chisels, 2, 4, 6, 8, 10 mm, Schwabe mastoied chisels, 2, 4, 6, 8, 10 mm, Defourmentel rongeur, straight 7½″ (19 cm), Cottle-Jansen rongeur, s-curved 7″ (18 cm), Luer rongeur, slightly curved 6″ (15 cm), Zaufal-Jansen rongeur with multiple action curved 7″ (18 cm), Hartmann's ear hook, sharp, Jacobson's ear hook, blunt, Lucae's ear hook, blunt, Mastoied curettes, Bishop's ear spoon, full sharp, Jansen's ear curettes, straight, sharp, Jansen's ear curettes, angled, sharp, Volkmann's ear curettes, Brun's ear curettes, Bowman's speculum 2¾″ (7 cm), Bowman's speculum, curved, Bowman's speculum with top screw, Graefe's speculum, McNamara's speculum, Knapp-Clark's speculum, Desmarres lid retractors, with fenestrated blades, Desmarres lid retractors, with solid blades, Eye scalpels, Graefe's cataract knives, Jaeger's keratomes, straight, Jaeger's keratomes, slightly angled, Jaeger's keratomes, full angled, Desmarre's corneal knifes, Critchet's canaliculus knife, Pool's corneal knife, Graefe's opthalmic knife, sickle shaped, Cataract needle, Cataract needle, Beer's cataract needle, Desmarre's paracentesis needle, Desmarre's paracentesis needle, Bowman's discission needle, Kuhnt's foreign body gouge, Rollet's chisel, Nicati foreign body chisel, Walton foreign body gouge, Walton foreign body gouge, Kuhnt's foreign body gouge, Graefe's iris hook sharp, Graefe's iris hook blunt, Graefe's cystitome, Guthrie's fixation hook, double, Axenfeld's iris hook, 2 prong, sharp, 2 prong, blunt, 3 prong, sharp, 3 prong, blunt, Rollet's hook, for lachrymal sac, Graefe's strabismus hook, blunt, Jaeger's strabismus hook, sharp, Wecker iris spatula, Meyhofer chalazion curette, Herba chalazion curette, Graefe's iris forceps, serrated, straight, slightly curved, strongly curved, 1×2 teeth, straight, slightly curved, strongly curved, Iris forceps, fluted handle, straight, Stevens iris forceps, serrated, Stevens iris forceps, 1×2 teeth, Graefe's iris forceps, 1×2 teeth, angled, Noyes fixation forceps, Noyes fixation forceps, with lock, Graefe's fixation forceps, Graefe's fixation forceps, with lock, Waldau fixation forceps, Elsching fixation forceps, Desmarres chalazion forceps, with set screw, Ayer chalazion forceps, Ayer chalazion forceps, with set screw, Heath chalazion forceps, with set screw, Lambert chalazion forceps, with set screw, Prince trachoma forceps, Knapp trachoma forceps, Snellen entropium forceps, McCallan capsule forceps, Kuhnt capsule forceps, Elsching capsule forceps, Iris scissors with flat shanks 4½″ (11 cm) straight, Iris scissors with flat shanks 4½″ (11 cm) curved on flat, Iris scissors with flat shanks 4½″ (11 cm) angular, Eye scissors, straight, pointed 4¾″ (12 cm), Eye scissors, curved, pointed 4¾″ (12 cm), Eye scissors, straight, blunt 4¾″ (12 cm), Eye scissors, curved, blunt 4¾″ (12 cm), Knapp strabismus scissors, straight, curved 4″ (10 cm), Strabismus scissors, straight curved 4¾″ (12 cm), Strabismus scissors, straight curved 4″ (10 cm), Noyes iris scissors, straight, 4¾″ (12 cm), Noyes iris scissors curved, 4¾″ (12 cm), Wecker iris scissors, 4½″ (11 cm), Wescott's iris scissors, sharp/sharp 4½″ (11 cm), Wescott's iris scissors, blunt/blunt 4½″ (11 cm), Iris dissecting scissors 3½″ (9 cm), Iris dissecting scissors 3½″ (9 cm) angular, blunt points, Iris dissecting scissors 3½″ (9 cm) straight, sharp points, Mathieu's iris scissors, straight 4½″ (11 cm), and Mathieu's iris curved 4½″ (11 cm).

A certain set of gall bladder instruments that can be sterilized in the disclosed sterilizers includes, Grey's gall duct forceps, serrated, B/J 8¾″ (22 cm), Grey's gall duct forceps, serrated, 1×2 teeth, box joint 8¾″ (22 cm), O'Shaugnessy gall duct forceps, serrated, box joint 7″ (19 cm), Shallcross gall duct forceps, box joint 7″ (18 cm), Crile gall duct forceps, screw joint 8¼″ (21 cm), Desjardin's gall bladder forceps, box joint 8″ (20 cm), Desjardin's gall stone forceps, screw joint, 8¼″ (21 cm), Mayo-Blake gall stone forceps, box joint 8″ (20 cm), Czerny gall stone forceps, screw joint 10″ (25 cm), Lister uretheral sounds, English scale 1 to 16, Metal catheters male, 1, 5 to 9 mm, Metal catheters male, angle 6 to 12, and Metal catheters female, 1, 5 to 9 mm.

A certain set of Gynecological instruments that can be sterilized in the disclosed sterilizers includes, Cusco's vaginal speculum, large plain (duck-bill), Medium plain (duck-bill), Small plain (duck-bill), Cusco's vaginal speculum, large, regular pattern with folding handles, Medium, regular pattern with folding handles, Small, regular pattern with folding handles, Large with winged screw, Medium with winged screw, Small with winged screw, Collin's vaginal speculum large with 4½″×1⅝″ blades, Medium 4¼″×1½″ blades, Small 3¾″×1¼″ blades, Grave's vaginal speculum large with 4½″×1⅜″ blades, Medium 4″×1¼″ blades, Small 3″×¾″ blades, Auvard's vaginal speculum with removable weight, Auvard's vaginal speculum weighted, Sims vaginal speculum, Eastman vaginal speculum, Doyen vaginal speculum, Doyen vaginal speculum with slightly concave blade, Doyen vaginal speculum with medium curved blade, large, medium, small, Sim's uterine depressors, Braun's uterine depressors, Hegar uterine dilators, set of 13 double ended ½ to 25/26 mm, Hegar uterine dilators 26, size, single ended with sloped handle, Goodell uterine dilators 13″ (33 cm), Wylie's uterine dilators with corrugated blades 12″ (30 cm), Sim's uterine dilators triple blade 12″ (30 cm), Kogan's endospeculum with scale-ratchet 8 fixing screws 9½″ (24 cm), Without ratchet 9½″ (24 cm), Simpson's uterine sounds, Martin's uterine sounds, Mayo's uterine sounds, Sim's uterine sounds, Schroeder's uterine curettes, Zweifel double ended curette with one sharp and one blunt end, Blake double ended curette with one sharp and one blunt end, Sim's uterine curette, Recamier's uterine curette, sharp/blunt, Greene uterine curette, Thomas uterine curette, blunt, round, Bozemann uterine dressing forceps, box joint 10″ (25 cm), Bozemann-Douglas uterine dressing forceps, box joint 10″ (25 cm), Bozemann uterine dressing forceps, box joint 10″ (25 cm), Fletcher sponge forceps, box joint, straight/curved 9½″ (24 cm), Foerster's sponge holding forceps, box joint, straight/curved plain jaws 7½″ (19 cm), 9½″ (24 cm), 10″ (25 cm), Foerster's sponge holding forceps, box joint, straight/curved plain/serrated jaws 7¼″ (19 cm), 9½″ (24 cm), 10″ (25 cm), Rampley's sponge holding forceps, box joint, straight/curved plain/serrated jaws 7½″ (19 cm), 9¼″ (24 cm), 10″ (25 cm), Maier's uterine polypus forceps, screw joint without ratchet, straight/curved 8″ (20 cm), Maier's uterine polypus forceps, screw joint without ratchet, straight/curved 10″ (25 cm), Maier's uterine polypus forceps, box joint, straight/curved 10″ (25 cm), Collin uterine forceps, collin lock 10″ (25 cm), Cheron uterine forceps, collin lock 10″ (25 cm), Skene tenaculum forceps, box joint 9″ (24 cm), Jarcho tenaculum forceps, box joint 8″ (20 cm), Schroeder's tenaculum forceps, box joint, Schroeder's tenaculum forceps with bent bows, box joint, Braun's tenaculum forceps, box joint 10″ (25 cm), Schroeder's enaculum forceps, box joint 10″ (25 cm), Pratt's vusellum forceps, screw joint 11″ (28 cm), Jacob's vusellum forceps, box joint 8¼″ (21 cm), Jacob's vusellum forceps, box joint, curved sideways 8¼″ (21 cm), Henrotin's vusellum forceps, box joint 8″ (20 cm), Teale's vusellum forceps, curved sideways 9″ (23 cm), Museux's vusellum forceps, screw joint straight 9½″ (24 cm) jaw width 6 mm, 9½″ (24 cm) jaw width 8 mm, 9½″ (24 cm) jaw width 10 mm, 9½″ (24 cm) jaw width 6 mm curved, 9½″ (24 cm) jaw width 8 mm curved, 9½″ (24 cm) jaw width 10 mm curved, 10½″ (26 cm) jaw width 9 mm straight, 10¼″ (26 cm) jaw width 11 mm straight, 10¼″ (26 cm) jaw width 13 mm straight, Museux's vusellum forceps, screw joint, curved 10¼″ (26 cm) jaw width 9 mm, 10¼″ (26 cm) jaw width 11 mm, 10¼″ (26 cm) jaw width 13 mm, dartigues uterine elevating forceps 9¾″ (25 cm), Somer's uterine elevating forceps 9½″ (24 cm), Wertheim hysterectomy forceps, box joint with one transverse tooth 7″ (28 cm), Mikulicz hysterectomy forceps, box joint with one transverse tooth 8″ (20 cm), Faure hysterectomy forceps, box joint with one transverse tooth 8¼″ (21 cm), Heaney hysterectomy forceps, box joint, single tooth 8¼″ (21 cm), Heaney hysterectomy forceps, box joint, double tooth 8¼″ (21 cm), Wertheim parametrium clamp, box joint 8½″ (22 cm), Wertheim-Cullen parametrium clamp, box joint 8½″ (22 cm), Fergusson's angiotribe forceps, box joint, straight 6¼″ (16 cm), 8″ (20 cm), Curved 6¼″ (16 cm), and Curved 8″ (20 cm).

A certain set of Intestinal instruments that can be sterilized in the disclosed sterilizers includes, Collin-Duval intestinal holding forceps, Allis (tissue) intestinal holding forceps B/J, 4×5 teeth 6″ (15 cm), 5×6 teeth 6″ (15 cm), 7½″ (19 cm), 9″ (23 cm), Thomas-Allis tissue intestinal holding forceps 8″ (20 cm) box joint, Duval intestinal holding forceps B/J, 8¼″ (21 cm), S/J, 7½″ (19 cm), B/J, 9″ (23 cm), Babcock intestinal holding forceps B/J 6¼″ (16 cm), Babcock intestinal holding forceps B/J 8″ (20 cm), B/J 9″ (23 cm), Lovelace's intestinal clamp forceps, box joint, straight 8″ (20 cm), Curved sideways 8″ (20 cm), Child's intestinal holding forceps, rubber jaw, box joint 10″ (25 cm), Doyen's intestinal clamp with longitudinal serrations S/J straight 9″ (23 cm), Curved 9″ (23 cm), Oblique serrations straight 9″ (23 cm), Oblique serrations curved 9″ (23 cm), B/J straight 9″ (23 cm), Curved 9″ (23 cm), Doyen's intestinal clamp longitudinal serrations, B/J straight 9″ (23 cm), Curved 9″ (23 cm), Kocher's intestinal clamp forceps, S/J straight/curved 8¾″ (22 cm), 9″ (23 cm), Kocher's intestinal clamp forceps, B/J straight/curved 8¾″ (22 cm), 10″ (25 cm), 11″ (28 cm), Moynihan's intestinal clamp forceps, S/J straight/curved 11½″ (29 cm), Payr's pylorus clamp with guide pin, large size 13¾″ (35 cm), Medium size 11½″ (29 cm), Small size 8¾″ (21 cm), Payr's pylorus without guide pin, large size 13¾″ (35 cm), Medium size 11½″ (29 cm), Small size 8¼″ (21 cm), and Lane's gastroenterostomy twin clamp 12″ (30 cm).

A certain set of Kidney Instruments that can be sterilized in the disclosed sterilizers includes, Randall's kidney stone forceps, 9″ (23 cm), Wertheim's kidney pedical clamp forceps, box joint, curved 10″ (25 cm), Wertheim-Cullen's kidney pedical forceps, box joint, curved 7½″ (19 cm), Morris kidney retractor 8¼″ (21 cm), and Kelly's kidney retractor 8¼″ (21 cm).

A certain set of Nasal Instruments that can be sterilized in the disclosed sterilizers includes, Voltolini nasal specula, Duplay nasal specula, Thudichum's (Goldsmith) nasal specula, Vienna nasal specula (US style), Vienna nasal specula (current), Tieck-Halle nasal specula, for infants, Hartmann-Halle nasal specula, Hartmann's nasal specula, Hartmann's nasal polypus forceps 6¼″ (16 cm), Hartmann's nasal polypus forceps 8¼″ (21 cm), Tilley nasal polypus forceps, serrated jaws 6¾″ (17 cm), Gross nasal polypus forceps, screw joint light model straight/curved without catch 5″ (13 cm), Gross nasal polypus forceps, screw joint, light model straight/curved 5½″ (14 cm), Gross nasal polypus forceps, screw joint, light model straight/curved, with catch 6¼″ (16 cm), Gross nasal polypus forceps, screw joint, light model straight/curved, with catch 5″ (13 cm), Gross nasal polypus forceps, screw joint, light model straight/curved, with catch 5½″ (14 cm), Gross nasal polypus forceps, screw joint, light model straight/curved, with catch 6¼″ (16 cm), Duplay's nasal polypus and dressing forceps, screw joint, straight 8″ (20 cm), Curved 8″ (20 cm), Luc septum cutting forceps, Bruenings septum cutting forceps, Weil-Blakesley nasal cutting forceps, straight, Weil-Blakesley nasal cutting forceps, curved upwards, Blakesley nasal cutting forceps, straight, Hartmann's nasal cutting forceps, Noye's (alligator) nasal polypus forceps, Joseph nasal scissors, straight, Heymann's nasal scissors, smooth blades, Cottle's nasa; scissors, for rhinoplasty, Choronshitzky nasal dressing forceps, serrated 6¼″ (16 cm), Choronshitzky nasal dressing forceps, 1×2 teeth, Troeltsch nasal dressing forceps 6¾″ (17 cm), 6¼″ (16 cm), Troeltsch nasal dressing forceps, 1×2 teeth, 6¾″ (17 cm), Ballenger swivel knife, and Ballenger swivel knife.

A certain set of Needle Holders that can be sterilized in the disclosed sterilizers includes, Mayo Hegar needle holder, box joint 5″ (13 cm), Mayo Hegar needle holder, box joint 6″ (15 cm), Mayo Hegar needle holder, box joint 7″ (18 cm), Mayo Hegar needle holder, box joint 8″ (20 cm), Crile Wood needle holder box joint 6″ (15 cm), Adson needle holder one fenestrated jaw box joint 7″ (18 cm), Collier needle holder box joint 5″ (13 cm), Masson needle holder straight broad jaw, box joint 10½″ (27 cm), Wangensteen needle holder straight narrow jaws 10½″ (27 cm), Finochietto needle holder curved jaws 10½″ (27 cm), Johnson needle holder double curved jaws 10½″ (27 cm), Mathieu needle holder for delicate sutures screw joint 5½″ (14 cm), Mathieu needle holder, jaws with groove/without groove, screw joint 5½″ (14 cm), 6¾″ (17 cm), 7¼″ (19 cm), 8″ (20 cm), Box joint 5½″ (14 cm), 6¾″ (17 cm), 7½″ (19 cm), 8″ (20 cm), Mathieu needle holder, jaws with groove/without groove, screw joint 5½″ (14 cm), 6¾″ (17 cm), 7½″ (19 cm), 8″ (20 cm), Box joint 5½″ (14 cm), 6¾″ (17 cm), 7½″ (19 cm), 8″ (20 cm), Olsen-Hegar needle holder & scissors combined, screw joint 5½″ (14 cm), 6½″ (16½ cm), 7¼″ (18½ cm), Gillies needle holder & scissors combined 6¼″ (16 cm), Langenbeck (Vienna Pattern needle holder, and box joint 6¼″ (16 cm), 7″ (18 cm), 8″ (20 cm).

A certain set of Obstretics Instruments that can be sterilized in the disclosed sterilizers includes, Collin Pelvimeters graduated in centimeters, Martin pelvimeters graduated in centimeters, Breisky pelvimeters graduated in centimeters, Simpson obstetrical forceps 12½″ (32 cm), Simpson obstetrical forceps 12½″ (32 cm), Kielland obstetrical forceps 15½″ (40 cm), Luikart-Kielland obstetrical forceps 15½″ (40 cm), Anderson's obstetrical forceps 15½″ (39 cm), Barne's obstetrical forceps 14″ (36 cm), Luikart-Simpson obstetrical forceps 14″ (36 cm), Piper obstetrical forceps 17½″ (44 cm), Neville-Barne's obstetrical forceps with traction handle 14″ (36 cm), 15½″ (39 cm), Milne-Murray's obstetrical forceps with traction handle 15½″ (39 cm), Tarnier obstetrical forceps with traction handle 15½″ (39 cm), Braun's cranioclast 16½″ (42 cm), Denman's (Smellie's) perforator screw joint 10″ (25 cm), Box joint 10″ (25 cm), McClintock ovum forceps, curved, screw joint 9″ (23 cm), Saenger placenta forceps, curved, without ratchet 11½″ (29 cm), 10½″ (27 cm) with ratchet, Umbilical scissors American pattern 4¼″ (11 cm), Dubois decapitation scissors, straight/curved 10½″ (27 cm), Oral and Tonsil Instruments, Heister's mouth gag, Doyen-Collin mouth gag, Doyen-Jansen mouth gag, Fergusson's mouth gag adult, Fergusson's mouth gag child, Fergusson-Ackland mouth gag with sliding ring, Mason-Ackland mouth gag child 5½″ (14 cm), Mason-Ackland mouth gag with sliding ring 8″ (20 cm), Mason-Ackland mouth gag with lock-tite ratchet 8″ (20 cm), Mason Ackland mouth gag with set screw 8″ (20 cm), Tongue spatula, Bosworth tongue depressor, Tobold tongue depressor, Andrews tongue depressor, Yankauer suction tube with additional tubing connection, Collin's tongue holding forceps, screw joint 6¼″ (16 cm), Young's tongue holding forceps, box joint 6¼″ (16 cm), Guly's tongue holding forceps, screw joint 7½″ (19 cm), Carmalt's tongue forceps, screw joint 6″ (15 cm), Colver's tonsil seizing forceps, screw joint, straight 7½″ (19 cm), Colver's tonsil seizing forceps, screw joint, curved 7½″ (19 cm), Ballenger's tonsil seizing forceps, box joint 8½″ (22 cm), Ballenger's tonsil seizing forceps, box joint 8½″ (22 cm), Tyding tonsil grasping forceps, box joint, Schnidt tonsil forceps, box joint, light curved, Schnidt tonsil forceps, box joint, full curved, Birkett tonsil forceps, box joint, straight, Birkett tonsil forceps, box joint, curved, Negu's tonsil artery forceps, screw joint small curved 7½″ (19 cm), Negu's tonsil artery forceps, screw joint large curved 7½″ (19 cm), Jackson tonsil forceps, box joint, Boettcher's tonsil scissors 7″ (18 cm), Tonsil scissors 7½″ (19 cm), Prince tonsil scissors 7″ (18 cm), Sluder-Ballenger tonsillectome, blade, Tobold's laryngeal polypus forceps, curved on flat, Fraenkel's laryngeal polypus forceps, Mackenzie's laryngeal polypus forceps, 9″ (23 cm), Chevalier Jackson's biopsy forceps, 12″ (30 cm), 16″ (40 cm), Chevalier Jackson's biopsy forceps, 11″ (28 cm), 21½″ (54 cm).

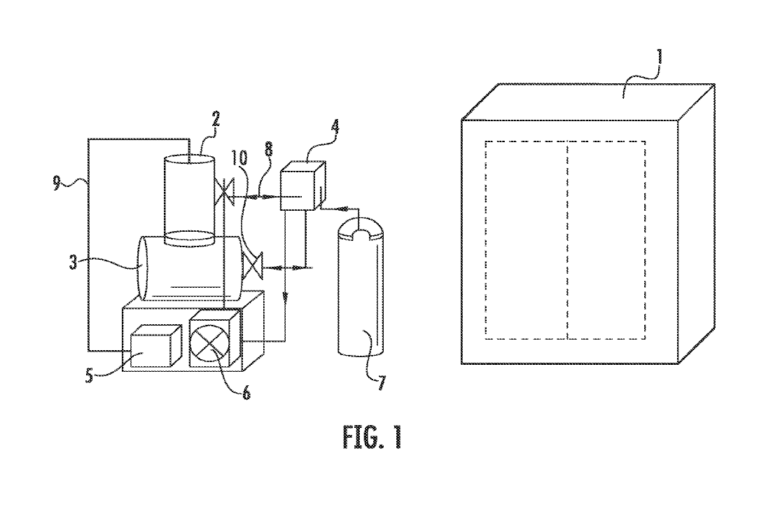

A certain set of Orthopedic Instruments that can be sterilized in the disclosed sterilizers includes, Osteotome (14 cm), Bone chisel with bevelled edge (14 cm), Brun's osteotome 8 mm wide 7½″ (19 cm), Brun's osteotome 10 mm wide 7½″ (19 cm), Brun's osteotome 12 mm wide 7½″ (19 cm), Brun's chisel 8 mm wide 7½″ (19 cm), Brun's chisel 10 mm wide 7½″ (19 cm), Brun's chisel 12 mm wide 7½″ (19 cm), McEwen's osteotome 8 mm wide 7½″ (19 cm), McEwen's osteotome 10 mm 7½″ (19 cm), McEwen's osteotome 12 mm wide 7½″ (19 cm), McEwen's osteotome 14 mm wide 7½″ (19 cm), McEwen's chisel, 5/16″ wide 7½″ (19 cm), McEwen's chisel 7/16″ wide 7½″ (19 cm) McEwen's chisel 9/16″ wide 7½″ (19 cm), McEwen's chisel ¾″ wide 7½″ (19 cm), McEwen's chisel 1″ wide 7½″ (19 cm), McEwen's chisel 1½″ wide 7½″ (19 cm), Doyen bone mallet, 8¼″ (21 cm), Hajek bone mallet 8¼″ (21 cm), Collin bone mallet 8″ (20 cm), Volkmann collin bone curettes, Simon's bone curettes, Volkmann bone curettes 5″ (13 cm), Volkmann bone curettes 5¾″ (14.5 cm), Volkmann bone curettes 6¾″ (17 cm), Volkmann bone curettes 8″ (20 cm), Bone curettes 8¼″ (21 cm), Williger bone curette, Hartmann's periosteal raspatory, Farabeur's periosteal raspatory 6″ (15 cm) left, Farabeuf's periosteal raspatory 6″ (15 cm) right, Doyen's costal periosteotome 6¾″ (17 cm) right, Doyen's costal periosteotome 6¾″ (17 cm) left, Lane's bone lever 10½″ (26.5 cm) with serrated end, Lambotte bone lever, Van buren sequestrum forceps B/J 9″ (23 cm), Sequestrum forceps 8″ (20 cm) screw/joint, straight/curved, Fergusson bone holding forceps 8¼″ (21 cm), Langenbeck bone holding forceps 8¼″ (21 cm), Lane's bone holding forceps 12½″ (31.6 cm), Lane's bone holding forceps 15½″ (39.3 cm), Lane's bone holding forceps 13″ (33 cm), Lane's bone holding forceps 17¾″ (45 cm), Farabeuf bone holding forceps 9″ (23 cm), Farabeuf bone holding forceps 10½″ (26 cm), Lambotte bone holding forceps 8¼″ (21 cm), Lambotte bone holding forceps 10″ (25 cm), Lambotte bone holding forceps 12″ (30 cm), Lambotte bone holding forceps 8¼″ (21 cm), Lambotte bone holding forceps 10″ (25 cm), Lambotte bone holding forceps 12″ (30 cm), Farabeuf lambotte bone holding forceps 10½″ (26 cm), Lowmann-Gerster bone clamp 7″ (18 cm), Lowmann-Gerster bone clamp 8″ (20 cm), Lowmann-Gerster bone clamp 8¾″ (22 cm), Luer's bone rongeur, screw joint 6″ (15 cm), Hartmann's bone rongeur, screw joint 6¾″ (17 cm), Luer's bone rongeur, box joint, straight/curved 7″ (18 cm), Luer's bone rongeur, box joint, straight/curved 6¾″ (17 cm), Stille bone rongeur, multiple action, straight 9″ (22½ cm), Stille bone rongeur, multiple action, curved on flat 9″ (22½ cm), Stille bone rongeur, multiple action, curved sideways 9″ (22½ cm), Chiron bone rongeur, multiple action, straight 7″ (18 cm), Chiron bone rongeur, multiple action, straight 8¾″ (22 cm), Stille bone rongeur, multiple curved sideways slender pattern 9″ (23 cm), Liston bone cutting forceps, screw joint, FIG. 1-3, 5½″ (14 cm), Liston bone cutting forceps, screw joint 6¾″ (17 cm), Liston bone cutting forceps, screw joint 7½″ (19 cm), Liston bone cutting forceps, screw joint 8¾″ (22 cm), Liston bone cutting forceps, double collin lock, 5½″ (14 cm), Liston bone cutting forceps, double collin lock, 6¾″ (17 cm), Liston bone cutting forceps, double collin lock, 7½″ (19 cm), Liston bone cutting forceps, double collin lock, FIG. 1-3, 8¾″ (22 cm), Liston bone cutting forceps, box joint, straight 5½″ (14 cm), Liston bone cutting forceps, box joint, straight 6¾″ (17 cm), Liston bone cutting forceps, box joint, straight 7½″ (19 cm), Liston bone cutting forceps, box joint, straight 8¾″ (22 cm), Liston bone cutting forceps, box joint, curved 5½″ (14 cm), Liston bone cutting forceps, box joint, curved 6¾″ (17 cm), Liston bone cutting forceps, box joint, curved 7½″ (19 cm), Liston bone cutting forceps, box joint, curved 8¾″ (22 cm), Liston bone cutting forceps, box joint, angular 5½″ (14 cm), 6¾″ (17 cm), 7½″ (19 cm), 8¾″ (22 cm), Littauer liston forceps, box joint 6″ (15 cm), Liston bone cutting forceps, multiple action, straight 10½″ (27 cm), Liston bone cutting forceps, multiple action, angled 10½″ (27 cm), Liston-Key bone cutting forceps, multiple action, double curved 10½″ (27 cm).

A certain set of Probes and directors that can be sterilized in the disclosed sterilizers includes, Probes double ended 4½″ (11½ cm), 5″ (13 cm), 5½″ (14 cm), 6″ (15 cm), 8″ (20 cm), 10″ (25 cm), Probes with chisel 4½″ (11½ cm), 5″ (13 cm), 5½″ (14 cm), 6″ (15 cm), 8″ (20 cm), 10″ (25 cm), Myrtle leaf probe 4½″ (11½ cm), 5″ (13 cm), 5½″ (14 cm), 6″ (15 cm), 8″ (20 cm), 10″ (25 cm), Probe with eye 4½″ (11½ cm), 5″ (13 cm), 5½″ (14 cm), 6″ (15 cm), 8″ (20 cm), 10″ (25 cm).

A certain set of Retractors that can be sterilized in the disclosed sterilizers includes, Grooved directors with tong tie 5″ (13 cm), 5½″ (14 cm), 6″ (15 cm), 8″ (20 cm), Grooved directors with tong tie 5″ (13 cm), 5½″ (14 cm), 6″ (15 cm), 8″ (20 cm), Grooved directors with tong tie & probe 5″ (13 cm), 5½″ (14 cm), 6″ (15 cm), 8″ (20 cm), Volkmann's retractor sharp pointed, 1 prong, Volkmann's retractor sharp pointed, 2 prong, Volkmann's retractor sharp pointed, 3 prong, Volkmann's retractor sharp pointed, 4 prong, Volkmann's retractor sharp pointed, 6 prong, Volkmann's retractor blunt pointed, 1 prong, Volkmann's retractor blunt pointed, 2 prong, Volkmann's retractor blunt pointed, 3 prong, Volkmann's retractor blunt pointed, 4 prong, Volkmann's retractor blunt pointed, 6 prong Kocher's retractor 1½″×½″ blade, Kocher's retractor 2″×½″ blade, Ollier's retractor ¾″×½″ (19 mm×13 mm) blade, Middledorpf's retractor, Middledorpf's retractor with hollow handle, Czerny's retractor 1¼″×⅞″ blade, Senn-Mueller retractor, Langenbeck's retractor 1¾″×½ blade, Senn green retractor (−6×20 mm), (−2.6×10 mm), Dissecting tenaculum 1 prong sharp with metal handle, Dissecting tenaculum 2 prong sharp with metal handle, Dissecting tenaculum double ended 2 prongs sharp, Durham retractor with hollow handle, Simon's retractor, 1 1/16″×4½″ blade, Doyen's retractor 1½″×3¼″ blade, Fritsch's retractor 40 mm blade, Fritsch's retractor 50 mm blade, Fritsch's retractor 60 mm blade, Fritsch's retractor 70 mm blade, Deaver retractor 7⅛″×1¾″ (18 cm×19 mm), Deaver retractor 8½″×⅞″ (21½ cm×22 mm), Deaver retractor 9″×1″ (23 cm×25 mm), Deaver retractor 12″×1½″ (30 cm×13 mm) Deaver retractor 12″×1″ (30 cm×25 mm), Deaver retractor 13″×1″ (33 cm×25 mm), Deaver retractor 14″×1½″ (36 cm×25 mm), Deaver retractor 12″×1½″ (30 cm×38 mm), Deaver retractor 12″×2″ (30 cm×50 mm), Finsen retractor 2×3 blunt prongs, Weitlaner retractor 3×4 prongs sharp or blunt 5″ (13 cm), Weitlaner retractor 3×4 prongs sharp or blunt 6½″ (16.5 cm), Weitlaner retractor 3×4 prongs sharp or blunt 5″ (13 cm), Weitlaner retractor 3×4 prongs sharp or blunt 6½″ (16.5 cm), Collin retractor with 2 lateral siwvel blades only, Collin retractor with 2 lateral swivel blades only and detachable central blade, Balfour retractor standard pattern lateral blades 58 mm, 2¼″ deep, spread 12 cm 4¾″, Farabeuf retractor 15 mm wide, Mayo collin retractor, and Parker retractor.

A certain set of Saws and Plaster instruments that can be sterilized in the disclosed sterilizers includes, Engel's plaster saw 6″ (15 cm), Bergmann's plaster saw, Kaulich's plaster saw 9″ (23 cm), Langenback's metacarpal saw with hollow handle 9″ (23 cm), blade 4½″ (11.5 cm), Charrier's bone saw with mobile back 8″ (20 cm), Charrier's bone saw with mobile back 10½″ (27 cm), Charrier's bone saw with mobile back 12″ (30 cm), Charrier's bone saw with mobile back 13½″ (34 cm), Satterle's bone saw 11¼″ (28.5 cm) with blade 8″ (20 cm), Weiss blade saw detachable, Esmarch plaster knife 7″ (18 cm), Bergmann plaster knife 7″ (18 cm), Reiner plaster knife 7″ (18 cm), Plaster knife with wooden handle, Stille's plaster shears, standard pattern 9″ (23 cm), Stille's plaster shears, standard pattern 10¼″ (26 cm), Stille's plaster shears, standard pattern 14½″ (37 cm), Stille's plaster shears 9″ (23 cm), Brun's plaster scissors, plain 8¾″ (22 cm), Seutin's plaster shears 9″ (23 cm), Brun's plaster scissors one blade serrated 8¾″ (22 cm), Bergmann plaster scissors 9″ (23 cm), Lorenz plaster scissors 9½″ (24 cm), Schulze gauze shears 8¼″ (21 cm), Smith gauze shears 7″ (18 cm), Knowles bandage scissors 5″ (13 cm), Knowles bandage scissors 5½″ (14 cm), Lister bandage scissors 3½″ (9 cm), Lister bandage scissors 4½″ (11½ cm), Lister bandage scissors 5½″ (14 cm), Lister bandage scissors 7¼″ (18½ cm), Hennig plaster spreader 11″ (28 cm), Wolff plaster cast breaker 7″ (18 cm), Wolff plaster cast breaker 9½″ (24 cm), Lister bandage scissors, one large ring 6½″ (16½ cm), Lister bandage scissors, one large ring 7¾″ (19 cm), and Lister bandage scissors, one large ring 8¼″ (21 cm).

A certain set of Scapels and operating knives that can be sterilized in the disclosed sterilizers includes, SCALPEL FORGED, SCALPEL FORGED, SCALPEL FORGED, DIEFFENBACH operating knives, BERGMANN operating knives, COLLIN operating knives, VIRCHOW, dissecting knives with wooden handle, SCALPEL HANDLE No. 4 for interchangeable blades, SCALPEL HANDLE No. 3 for interchangeable blades, CATLIN'S AMPUTATING KNIFE 5″ (13 cm), CATLIN'S AMPUTATING KNIFE 6¼″ (16 cm), CATLIN'S AMPUTATING KNIFE 7½″ (19 cm), CATLIN'S AMPUTATING KNIFE 8¾″ (22 cm), LISTON'S AMPUTATING KNIFE 5½″ (14 cm), LISTON'S AMPUTATING KNIFE 6¾″ (17 cm), LISTON'S AMPUTATING KNIFE 8″ (20 cm), COLLIN'S AMPUTATING KNIFE 4¼″ (12 cm), COLLIN'S AMPUTATING KNIFE 6″ (15 cm), COLLIN'S AMPUTATING KNIFE 7″ (18 cm), COLLIN'S AMPUTATING KNIFE 8¾″ (22 cm), CATLIN'S AMPUTATING KNIFE 5¼″ (14 cm), CATLIN'S AMPUTATING KNIFE 6¾″ (17 cm), CATLIN'S AMPUTATING KNIFE 8″ (20 cm), LANGENBECK'S AMPUTATING KNIFE 4¾″ (12 cm), LANGENBECK'S AMPUTATING KNIFE 5¼″ (13½ cm), ESMARCH RESECTION AMPUTATING KNIFE, and LANGENBECK RESECTION AMPUTATING KNIFE.

A certain set of Scissors that can be sterilized in the disclosed sterilizers includes, Operating scissors, straight, sharp/blunt, blunt/blunt, sharp/sharp, pattern A.B.C. 4½″ (11½ cm), 5″ (13 cm), 5½″ (14 cm), 6″ (15 cm), 7″ (18 cm), 8″ (20 cm), Operating scissors, curved, sharp/blunt, blunt/blunt, sharp/sharp, pattern A.B.C. 4½″ (11½ cm), 5″ (13 cm), 5½″ (14 cm), 6″ (15 cm), 7″ (18 cm), 8″ (20 cm), Operating scissors, open flat, sharp/sharp 5″ (13 cm), Operating scissors, probe pointed 5″ (13 cm), 5½″ (14 cm), 6½″ (16½ cm), Dissecting scissors 4½″ (11 cm), 5″ (13 cm), 5½″ (14 cm), Lister bandage scissors 3½″ (9 cm) 4½″ (11½ cm), 5½″ (14 cm), 7½″ (18½ cm), Mayo-Stille operating scissors, straight 5½″ (14 cm), 6¼″ (15½ cm), 6¾″ (17 cm), 9″ (23 cm), Curved, 5½″ (14 cm), 6¼″ (15½ cm), 6¾″ (17 cm), 9″ (23 cm), Mayo operating scissors, straight 5½″ (14 cm), 6¼″ (15½ cm), 6¾″ (17 cm), 9″ (23 cm), Mayo operating scissors, curved 5½″ (14 cm), 6¼″ (15½ cm), 6¾″ (17 cm), 9″ (23 cm), Mayo operating scissors, straight 5½″ (14 cm), 6¼″ (15½ cm), 6¾″ (17 cm), 9″ (23 cm), Mayo operating scissors, curved 5½ ″ (14 cm), 6¼″ (15½ cm), 6¾″ (17 cm), 9″ (23 cm), Mayo operating scissors, straight 5½″ (14 cm), 6¼″ (15½ cm), 6¾″ (17 cm), 9″ (23 cm), Mayo operating scissors, curved 5½″ (14 cm), 6¼″ (15½ cm) 6¾″ (17 cm), 9″ (23 cm), Mayo-Lexer operating scissors, straight 6¼″ (16 cm), Mayo-Lexer operating scissors, curved 6¼″ (16 cm), Doyen surgical scissors, straight 7″ (18 cm), Doyen surgical scissors, curved 7″ (18 cm), Smith bandage scissors 7″ (18 cm), Richter surgical scissors angled sideways, S/B, B/B, S/S 5″ (13 cm), Richter surgical scissors angled sideways S/B, B/B, S/S 5¾″ (14 CM), Epistotomy scissors angled laterally 5″ (13 cm), Sims uterine scissor 8″ (20 cm) straight S/B, B/B, S/S, Sims uterine scissor 8″ (20 cm) curved S/B, B/B, S/S, Sims uterine scissor 8″ (20 cm) straight S/B, B/B, S/S, Sims uterine scissors 8″ (20 cm) curved S/B, B/B, S/S, Wertheim scissors straight 8″ (20 cm), 9″ (23 cm), Wertheim scissors slightly curved 8″ (20 cm), 9″ (23 cm), Metzenbaum operating scissors 7″ (18 cm), 8″ (20 cm), Metzenbaum operating scissors curved 7″ (18 cm), 8″ (20 cm), Kelly's vascular scissors straight 6¼″ (16 cm), Curved 6¼″ (16 cm), Maier's scissors 6″ (15 cm) Maier's scissors 7″ (18 cm), Smith's scissors 7″ (18 cm), Smith's scissors 8″ (20 cm), Smith's scissors 9″ (23 cm), Ward scissors, heavy 7″ (18 cm), 8″ (20 cm), 9″ (23 cm), Sinus and drainage forceps, Sinus forceps, screw joint 5″ (13 cm), 6″ (15 cm), 7″ (18 cm), 8″ (20 cm), Sinus forceps, box joint 5″ (13 cm), 6″ (15 cm), 7″ (18 cm), 8″ (20 cm), Bryant's dressing forceps, screw joint 5″ (13 cm), Bryant's dressing forceps 5″ (13 cm), French pattern dressing forceps, screw joint 5″ (13 cm), Kronleins dressing forceps, box joint 5″ (13 cm), Maiers dressing forceps, screw joint 8″ (20 cm) Maiers dressing forceps, screw joint 10″ (25 cm), Gross dressing forceps, screw joint 5″ (13 cm), 5½″ (14 cm), 6″ (15 cm), 7″ (18 cm), 8″ (20 cm), Splinter forceps, Splinter forceps, very fine points, straight/curved without guide pin, 3½″ (9 cm), 4½″ (11½ cm), 5″ (13 cm), Splinter forceps, very fine points, straight/curved with guide pin 3½″ (9 cm), 4″ (11½ cm), 5″ (13 cm), Carmalt's splinter forceps, straight 4½″ (11½ cm), Curved 4½″ (11½ cm), Feilchenfeld forceps 3½″ (9 cm), 4½″ (11½ cm), 5″ (13 cm), Walter's splinter forceps, straight 4¼″ (11 cm), Curved 4¼″ (11 cm), Hunter's splinter forceps, straight 4½″ (11½ cm), Curved 4½″ (11½ cm), Arthur's splinter forceps, box joint, straight 5″ (13 cm), Curved 5″ (13 cm), Straight 5½″ (14 cm), Curved 5½″ (14 cm), Stieglitz's splinter forceps, box joint, straight 5½″ (14 cm), Curved 5½″ (14 cm), and Ralk's splinter forceps, box joint 5½″ (14 cm).

A certain set of Sterilizer instruments that can be sterilized in the disclosed sterilizers includes, Davis sterilizer forceps 6″ (15 cm), Davis sterilizer forceps 10″ (25 cm), Davis sterilizer forceps 7″ cross action (18 cm), Davis sterilizer forceps 10″ cross action (25 cm), Sterilizer forceps, 3 prongs 8″ (20 cm), Sterilizer forceps, 3 prongs 12″ (30 cm), Cheatle's sterilizer forceps, screw joint 10½″ (27 cm), Box joint 10½″ (27 cm), Heavy pattern 11″ (28 cm), Harrison's bowl sterilizer forceps, screw joint 10″ (25 cm), 12″ (30 cm), 14″ (35 cm), 18″ (45 cm), Bunt's holder for sterilizer forceps & scissors 5½″ (14 cm), 4¾″ (12 cm).

A certain set of Suture instruments that can be sterilized in the disclosed sterilizers includes, Childe's approximation forceps 7″ (18 cm), Childe's approximation forceps, with clip holder 7″ (18 cm), Championniere approximation forceps, straight 5½″ (13½ cm), Championniere approximation forceps, curved 5½″ (13½ cm), Wachenfeldt clip applying forceps 4¾″ (12 cm), Michel's clip applying forceps 4¾″ (12 cm), Michel's clip applying forceps 4¾″ (12 cm), Farkas clip applying forceps 5″ (12½ cm), Heath's clip removing forceps 5″ (13 cm), Michel clip applying and removing forceps, box joint 4¾″ (12 cm), Michel clip applying and removing forceps, box joint 4¾″ (12 cm), Lutz Michel clip removing hook 6″ (15 cm), Littauer's ligature scissors 5½″ (14 cm), Heath's ligature scissors 6″ (15 cm), Reverdin needles, small pattern, Reverdin needles, standard pattern, Reverdin needles, large pattern, Cooper's ligature needles, 7½″ (19 cm), Kocher's ligature needles, 7½″ (19 cm), Deschamp's ligature needles, and right or left handle 8″ (20 cm).

A certain set of Thoraic and lung surgery that can be sterilized in the disclosed sterilizers includes, Bone shears 9″ (23 cm), Rib shears 8¾″ (22 cm), Collin rib shears 8″ (20 cm), Gluck rib shears 7½″ (19 cm), Gluck rib shears 8¾″ (22 cm), Robert's rib shears 12¼″ (31 cm), Bethune's rib shears 13½″ (22 cm), Coryllo's rib shears left 13¾″ (35 cm), Coryllo's rib shears right 13¾″ (35 cm), Willauer lobectomy scissors, slightly curved 10¾″ (27 cm), Crafoord lobectomy scissors slightly curved 12″ (30 cm), Nelson lobectomy scissors straight/curved 10″ (25 cm), Nelson lobectomy scissors straight/curved 12″ (30 cm), Finochietto lobectomy scissors angled on flat 10¼″ (26 cm), Tuttle thoracic thumb forceps 9″ (23 cm), Nelson thoracic thumb forceps 6×7 teeth 9″ (23 cm), Mayo harrington thumb forceps 13¾″ (35 cm), Wangensteen needle holder, box joint 10½″ (27 cm), Finochietto needle holder, box joint 10½″ (27 cm), Johnson needle holder, box joint 10½″ (27 cm), Duval lung forceps, screw joint 8″ (20 cm), Duval lung forceps, box joint 8″ (20 cm), Duval collin lung forceps, box joint, 8″ (20 cm), Lahey artery forceps, box joint 9″ (23 cm), Davidson pulmonary vessel clamp, box joint 9″ (23 cm), Finochietto artery and ligature forceps, box joint 9½″ (24 cm), Roberts lung forceps, box joint, straight/curved 8¾″ (22 cm), Roberts lung forceps, box joint, straight/curved 9½″ (24 cm), Mixture thoracic forceps, box joint, 8¾″ (22 cm), Mixture thoracic forceps, box joint, 10″ (25 cm), Mixture thoracic forceps, box joint, 11″ (28 cm), Price-Thomas bronchus clamp, box joint 8¾″ (22 cm), Satinsky cardiovascular clamp, box joint, 10¾″ (27 cm), Brock cardiovascular clamp, box joint, 9″ (23 cm), Pott's clamp, box joint, 6½″ (16½ cm), and Pott's clamp, box joint, 8½″ (21½ cm).

A certain set of Thumb dressing and tissue forceps that can be sterilized in the disclosed sterilizers includes, Thumb dressing forceps, broad point 4½″ (11½ cm), 5″ (13 cm), 5½″ (14 cm), 6″ (15 cm), 7″ (18 cm), 8″ (20 cm), 10″ (25 cm), 12″ (30 cm), Thumb dressing forceps, fluted handle 4½″ (11½ cm), 5″ (13 cm), 5½″ (14 cm), 6″ (15 cm), 7″ (18 cm), 8″ (20 cm), 10″ (25 cm), 12″ (30 cm), Thumb dressing forceps, fine point, curved 5″ (13 cm), Thumb dressing forceps, turnover end 5″ (13 cm), 6″ (15 cm), 7″ (18 cm), 8″ (20 cm), 10″ (25 cm), 12″ (30 cm), Semkin's dressing forceps, delicate 5″ (13 cm), Chiron's dressing forceps 5″ (13 cm), Chiron's dressing forceps 5½″ (14 cm), Tissue forceps, 1×2 teeth 4½″ (11½ cm), 5″ (13 cm), 5½″ (14 cm), 6″ (15 cm), 7″ (18 cm), 8″ (20 cm), Tissue forceps, 2×3 teeth 5″ (13 cm), Tissue forceps, 2×3 teeth 5½″ (14 cm), Tissue forceps, 2×3 teeth 6″ (15 cm), Tissue forceps, 3×4 teeth 5″ (13 cm), Tissue forceps, 3×4 teeth 5½″ (14 cm), Tissue forceps, 3×4 teeth 6″ (15 cm), Tissue forceps, 1×2 teeth, fluted handle 4½″ (11½ cm), 5″ (13 cm), 5½″ (14 cm), 6″ (15 cm), Treves tissue forceps, 1×2 teeth, delicate/heavy pattern 5″ (13 cm), 5½″ (14 cm), 6″ (15 cm), 7″ (18 cm), Gillies tissue forceps, 1×2 teeth 6″ (15 cm), McIndoe dressing forceps 6″ (15 cm), Dressing forceps with flat end, Swedish pattern 5″ (13 cm), 6″ (15 cm), 6½″ (17 cm), 8″ (20 cm), Adson dressing forceps, serrated 4¾″ (12 cm), Adson dressing forceps, 1×2 teeth 4¾″ (12 cm), Bonney tissue forceps 7″ (18 cm), Adlerkreutz tissue forceps, narrow/broad 5″ (13 cm), 6″ (15 cm), 8″ (20 cm), Lane tissue forceps, 1×2 teeth 5½″ (14 cm), Lane tissue forceps, 1×2 teeth 7″ (18 cm), Lane tissue forceps, 2×3 teeth 5½″ (14 cm), Lane tissue forceps, 2×3 teeth 7″ (18 cm), Lane tissue forceps, 3×4 teeth 5½″ (14 cm), Lane tissue forceps, 3×4 teeth 7″ (18 cm), Duval tissue forceps, 5¾″ (14½ cm), Tuttle tissue forceps 7″ (18 cm), Tuttle tissue forceps 9″ (23 cm), Tissue forceps, and Russian Pattern 6″ (15 cm), 8″ (20 cm), 10″ (25 cm).

A certain set of Towel holding forceps that can be sterilized in the disclosed sterilizers includes, Jone's towel forceps cross action 3½″ (9 cm), Schadel's towel forceps cross action 3½″ (9 cm), Towel forceps, english pattern cross action 3½″ (9 cm), Doyen's towel forceps with ring 7″ (18 cm), Roeder's towel forceps, box joint 5¼″ (13½ cm), Mayo's towel forceps, box joint 5½″ (14 cm), Backhaus towel forceps, box joint 3½″ (9 cm), Backhaus towel forceps, box joint 4½″ (11½ cm), Backhaus towel forceps, box joint 5¼″ (13½ cm), Moynihan's towel forceps, screw joint 7½″ (19 cm), and Moynihan's towel forceps, box joint 7½″ (19 cm),

A certain set of Traechyotomy instruments that can be sterilized in the disclosed sterilizers includes, Trousseau's tracheal dilator 5¾″ (14½ cm), Chevalier-Jackson's tracheal tubes with pilot one inner tube 16 to 34 french gauge, 10 sizes normal radius, Magill's catheter introducing forceps, adult 10″ (25 cm), Child 8″ (20 cm), Senn-Miller tracheal retractors, and double ended, Senn-Green tracheal retractors.

In one embodiment of the disclosed sterilizer, the targeted item and the electron emitter are both located in the same low pressure atmosphere, which permits the use of a comparatively low voltage electrons of 30 keV, or less, rather than the customary voltage of 6 MeV or more. The higher voltages are required in sterilizers that operate on targeted items that are not located in a low pressure atmosphere. The higher voltages are needed to compensate for the loss of energy that the electrons encounter when the electrons leave device and enter the higher pressure atmosphere containing the targeted item. In the disclosed sterilizers, the electrons may optionally be rapidly moved and/or the targeted item may be shaken or to help expose all surfaces to the electrons. The disclosed sterilizers can adequately reduce bioburden of the targeted items with an electrons that is active for less than 3 minutes, which results in a full sterilizing cycle time of under 4 minutes. In certain embodiments, the electrons can be active for example, for less than or equal to or greater than 1 second, 5 seconds, 10 seconds, 20 seconds, 30 seconds, 45 seconds, 1 minute, 2 minutes, 3 minutes, 4 minutes, 5 minutes, 6 minutes, 7 minutes, 8 minutes, 9 minutes, 10 minutes, 15 minutes, 20 minutes, 50 minutes, and/or 100 minutes. The full sterilizing cycle time can be, for example, less than or equal to or greater than 1 second, 5 seconds, 10 seconds, 20 seconds, 30 seconds, 45 seconds, 0.2 minutes, 0.5, minutes, 0.8 minutes, 1 minute, 2 minutes, 3 minutes, 4 minutes, 5 minutes, 10 minutes, 15 minutes, or 20 minutes longer than the electrons active time. The relationship between electrons and time is controlled by the ability to handle heat of the bombardment of the electrons on the material to be sterilized. Different materials can handle the bombardment in different capacities, and thus, different materials can handle different lengths of time or degrees for bombardment. The length of time is a balance between the heat characteristics of the material, the flux of the electrons, the desired level of sterilization, and the desired end temperature. In certain embodiments, such as a compact version of the sterilizer, the targeted item can be held in a basket that is formed from a section of the vacuum chamber itself.

Steering coils in the sterilizer can alter the direction and diameter of the electrons. By varying these two parameters it is possible to steer the beam to achieve complete coverage of the targeted instrument or to constrict the beam such that it may be directed through a channel or lumen in the instrument. Alternatively, electrons can also be accelerated and when striking the object to be sterilized or some other part of the sterilizer, they can have their direction altered and electrons can be bounced and have more than one sterilization event be produced by each.

Additional sterilization is produced by the backscattered electrons which can freely travel in the vacuum. It should be noted that typical high energy electron sterilizers of the MeV variety do not generate useable quantities of backscattered electrons and those that are created are rendered ineffective though losses due to absorption in atmospheric pressure. The sterilizer can deliver a minimum of 25 kGray, or a set or required sterilizing dose, to the surface of a targeted item in a 3 minute cycle, or less, such as 2 minutes, 1 minutes, or less. The use of a coating with a high z value, such as 50, 49, 48, 47, or greater, on the inside of the vacuum chamber increases the efficiency of the sterilizer due to their production of greater number of backscattered electrons. The z value is the atomic number. These coatings can be used to coat the inside of the chamber, such as product chamber.

An alternate embodiment, instead of a high Z coating, would be to use electric field to redirect electrons from the walls of the chamber. This technique would efficiently recycle electrons back to the object to be sterilized, it would however prevent or reduce the electrons ability to sterilize the walls of the chamber. As an example, the chamber would be held at a ground potential and the target to be treated would be suspended in the chamber in a metal mesh bag held at a positive 25 kV potential. A source of electrons such as a filament at ground potential would generate free electrons which would be accelerated towards the bag. Any electrons passing through the bag without making contact would be de-accelerated, turned around, and re-accelerated towards the bag until ultimately striking the product. Similarly any backscattered electron resulting from such a collision would ultimately be turned by the electric field and result in additional interactions with only the product.

Certain coatings can enhance backscatter of the electrons. For example, coatings made from elements of atomic numbers greater than, for example, 40, 41, 42, 43, 44, 45, 46, 47, 48, 49, 50, 51, 52, 53, 54, 55, 56, 57, 58, 59, or 60. Preferred coatings can be made from Cr (24), Ag (47), and Au (79). In addition, density can have some effect as well, with higher densities Increasing reflection. For example, the layer of material should be thick enough to stop the electrons at the chosen electron energy.

Also the angle at which an electron hits a surface affects the probability of backscatter electrons being produced and the energy of those electrons. Generally a glancing blow will result in less loss in energy and greater probability of scattered electrons which will generally will continue in the direction of the initial electron. Electron hitting a surface at a normal angle will have the least probability of scatter, the electrons will lose more energy and will be turned in an opposite direction to the initial impact. These behaviors of electrons are dominant at low energies and almost non-existent at high energies (e.g., the several MeV energy levels used in commercial sterilizers). These behaviors allow low energy electrons to effectively be turned without electric fields or deflection coils for normal incident interactions. For shallow angle interactions the low energy electrons are able to bounce numerous times allowing them to penetrate into cracks and crevices with considerable efficiency.

Another embodiment of the sterilizer is particularly adapted for using electrons as a topical therapy to reduce and treat site infections, or potential infections, on live subjects like AIDS skin lesions, decubitus ulcers, and burns, for example. Irradiation is a known methodology for reducing bacterial and virus in cadaver transplant materials, medical products and food. However, because of the inherent fear of “radiation” very little clinic investigation beyond tumor therapy exists. One company has developed a low-energy, localized x-ray irradiation device and has failed (Photoelectron Corp), trying to use it for reduction of intracranial tumors. The disclosed sterilizer delivers a high dose of radiation to a very shallow depth by using the principles of low energy sterilization discussed herein. The electrons emitted will kill bacteria on the surface of cells or surrounding patient, eukaryotic cellular material, but will not penetrate or harm the eukaryotic cells.

In certain embodiments, the energy of the electrons is measured at the point of contact with the material or product to be irradiated. When measured this way, this energy, the effective energy, is determined or predetermined In certain embodiments, the effective energy can be at least, equal to, or less than, 1 keV, 2 keV, 3 keV, 4 keV, 5 keV, 6 keV, 7 keV, 8 keV, 9 keV, 10 keV, 12 keV, 13 keV, 14 keV, 15 keV, 16 keV, 17 keV, 18 keV, 19 keV, 20 keV, 21 keV, 22 keV, 23 keV, 24 keV, 25 keV, 26 keV, 27 keV, 28 keV, 29 keV, 30 keV, 31 keV, 32 keV, 33 keV, 34 keV, 35 keV, 36 keV, 37 keV, 38 keV, 39 keV, 40 keV, 41 keV, 42 keV, 43 keV, 44 keV, 45 keV, 46 keV, 47 keV, 48 keV, 49 keV, 50 keV, 55 keV, 60 keV, 65 keV, 70 keV, 75 keV, 80 keV, 85 keV, 90 keV, 95 keV, or 100 keV. Optionally, an electron energy can be sufficiently low such that the electrons do not produce a plasma. It should be understood that this energy level is related to the level of vacuum, e.g., at lower vacuum (e.g., lower absolute pressure) the electron energy can be greater without producing a plasma. Optionally, an electron energy of 25 keV or less can be effective for sterilizing medical instruments, with appropriate atmospheric conditions as described herein. Optionally, an electron energy level of less than 20 keV, 15 keV and 10 keV can be effective for sterilizing medical instruments, with appropriate atmospheric conditions as described herein. The energy of the electrons striking the surface irrespective of the initial electron acceleration voltage can be determined though analysis of the spectrum of the x-rays produced.

In certain embodiments, the device can be operated in a partial vacuum that permits any static charge that may have accumulated around items, such as plastic items, ceramic items, or non-conducting items to be discharged. For example, within an atmosphere of 50 mTorr, such static charges can be dissipated due to the ionization of the residual gases. If an atmosphere is less than 10⁻⁵ Torr, other avenues for dissipation of static charges, such as grounding the product can be used. Optionally, as discussed above, the atmosphere can be evacuated to other levels of vacuum such as 10⁻² Torr, 10⁻³ Torr, 10⁻⁴ Torr, 10⁻⁵ Torr, 10⁻⁶ Torr, etc., for example.

The method for delivering electrons to the skin or lesion surface without detrimental side effects uses a low-energy electrons projected through a low resistance path to the subject's target tissue area. While a low atmosphere path could be established to transfer the low energy electrons, too much vacuum can damage tissue. Therefore, it is preferred to use a low density inert gas, such as helium, to minimize energy loss as the electrons are transmitted to the target site. In such atmospheres, 10 keV electrons can travel considerably less than one millimeter before losing their sterilizing energy. In a helium atmosphere, the effective distance over which electrons can travel with sterilizing effect is multiple millimeters. The key point is that control of the energy and hence penetration of the electrons at the surface being treated can be precisely controlled by measuring the spectrum of emitted x-ray from the surface, and by using a gas shield with one component. Helium and hydrogen, due to their low density, offers the best penetration of electrons to the surface, the electrons passing though the gas uniformly and predictably lose energy so that the energy at the emitter can be increased to account for these losses. Argon could also be used but has the disadvantage that it would require significantly high initial acceleration voltages and that the Argon itself would be the source of high levels of fluorescence x-rays which, at ˜3 keV could contribute to an unacceptable dose of x-rays. Normal atmosphere due to the numerous component gases would cause the initial emitted electrons to decay in to a wide spectrum of energenic electrons at the surface being treated.

It is understood that the electron emitter can also be mechanically moved to scan, for example, the product.

It is also understood that the devices and methods can be operated at, for example, greater than or equal to, equal to, less than or equal to, greater than, or less than 75 μA, 150 μA, 300 μA, 500 μA, 750 μA, 1 mA, 5 mA, 10 mA, 25 mA, 50 mA, 75 mA, 100 mA, 250 mA, 500 mA, 1 A.

It also understood that the devices and methods can be operated at, for example, greater than or equal to, equal to, less than or equal to, greater than, or less than 100 kV, 80 kV, 60 kV, 50 kV, 40 kV, 30 kV, 25 kV, 20 kV, 15 kV, 10 kV, or 5 kV.

In is also understood that the devices and methods can be operated such that the beam is not focused to greater than or equal to, equal to, less than or equal to, greater than, or less than, to 500 mm, 400 mm, 300 mm, 200 mm, 100 mm, 90 mm, 80 mm, 70 mm, 60 mm, 50 mm, 40 mm, 30 mm, 20 mm, 10 mm, 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.08 mm, 0.06 mm, 0.04 mm, or 0.02 mm.

It is also understood that the devices and methods can be operated such that the surface of the product to be hit with electrons is larger than 0.1 cm,² 0.2 cm,² 0.3 cm,² 0.4 cm,² 0.5 cm,² 0.6 cm,² 0.7 cm,² 0.8 cm,² 0.9 cm,² 1 cm,² 2 cm,² 3 cm,² 4 cm,² 5 cm,² 6 cm,² 7 cm,² 8 cm,² 9 cm,² 10 cm,² 30 cm,² 50 cm,² 75 cm,² 100 cm,² 400 cm,² or 1000 cm².

The electron source can produce a quantity of electrons which can have a variable shape, such a focused shape, approximately a pencil shape for example, or dispersed shape, approximately a cone shape. The electrons can be produced in such a way as to produce a field or a beam. In a typical situation, a focused shape is a more concentrated electron quantity per area, and a cone shape is a more diffuse electron quantity per area. The shape of the electron quantity is related to the amount of sterilization desired, as well as the way in which the item to be sterilized is more or less uniformly sterilized. For example, if the quantity of electrons produced is in the shape of pencil have an area of coverage smaller than the item to be sterilized this electron quantity could be indexed and moved over the item, whereas if the quantity of electrons was more in the shape of a cone, the quantity might not need to be scanned, but the dose uniformity issues, for example, the edges relative to the center could be accounted for. For example, electrons produced in a field, will cause electrons to come from different vectors at different velocities. This can occur, for example, through reflection of electrons within the chamber.

The sterilizer system shown in FIG. 1 comprises a cabinet housing 1, which is the overall housing for the unit. The cabinet housing 1 can be made from any material, such as sheet metal. The cabinet housing 1 typically would contain an electron chamber 2, a product chamber 3, a vacuum gas manifold 4, a high voltage power supply 5, a vacuum pump 6, an inert gas cylinder 7 containing inert gas (e.g., helium), a vacuum pipe 8, high voltage cable 9, and gas valve 10. An electron emitter, not shown, would be located in electron chamber 2.

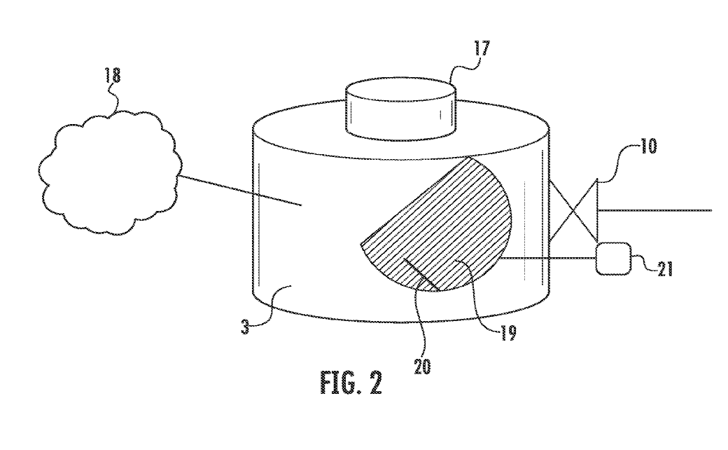

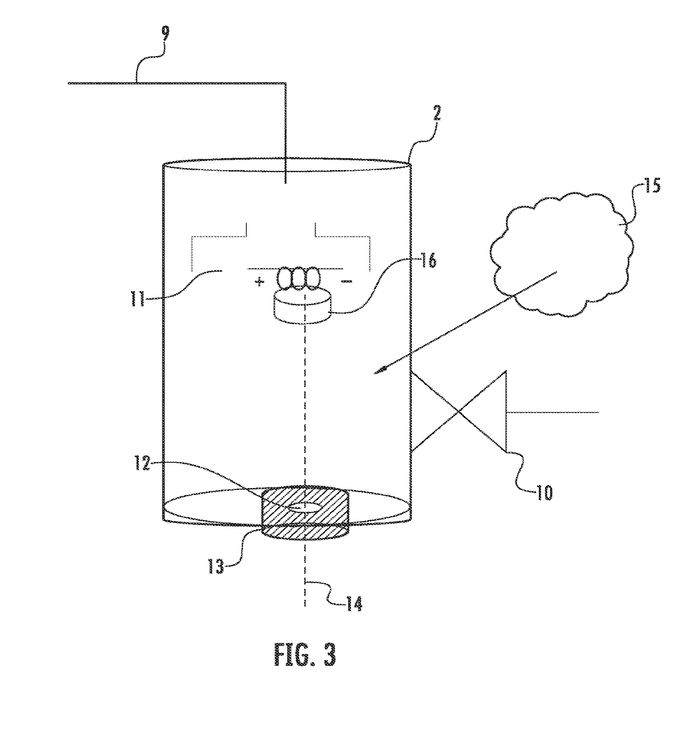

FIG. 2 illustrates the product chamber 3 with a coupler 17 that is adapted to connect electron chamber 2 to the product chamber 3. Coupler 17 mates with a complementary coupler on the electron chamber (not shown in FIG. 1). For the sterilizer shown in FIGS. 1 and 2, the electron chamber 2 and product chamber 3 have separate atmospheres for which pressure and composition may be controlled independently. Thus, atmosphere 18 in product chamber 3 may be sterilized dry air, nitrogen, an inert gas such as helium, or any other suitable gas at a suitably low pressure, preferably at least a partial vacuum. A concave circular container 19 for holding the targeted item is shown within product chamber 3 and may be made, e.g., of stainless steel. This disclosure also contemplates other types of product/target containers including containers that are not concave. An agitator 20 is located within the container 19 and the container 19 is rotated via a shaft connected to a motor 21. As motor 21 rotates the container, agitator 20 disrupts the targeted item so that the item is exposed more evenly and thoroughly to the electrons. Such an agitator 20 is particularly well suited to ensure that certain types of targeted items like powder, small granular material, or even small metallic or plastic parts, such as screws etc. are sufficiently exposed to the sterilizing effect of the electrons emitted by the emitter 11 shown in FIG. 3. Valve 10 coupled to gas cylinders and/or vacuum machinery is one mechanism for controlling the atmosphere 18 within product chamber 3. The temperature of the targeted item can be regulated to reduce emissions of water vapor and volatiles as needed by controlling the duration and strength of the electrons impinging the targeted item. Reducing such emissions is desirable to avoid contaminating atmosphere 18.

FIG. 3 schematically depicts electron chamber 2 containing atmosphere 15 and various operative elements. For example, electron emitter 11 is shown in electron chamber 2. The electron emitter 11 may be a filament, cathode dispenser, nanotube, or other known device for generating electrons. The electrons 14 are shown passing out of electron chamber 2 through aperture 12 and coupler 13, which is adapted to connect to coupler 17 from FIG. 2, on its way to entering product chamber 3 (also shown in FIG. 2). It is understood that in certain embodiments, the product chamber 3 and electron chamber 2 are connected in such a manner that they share a single atmosphere at all times. However, in the embodiment shown in FIGS. 1-3, there is a coupling connector 13, which connects electron chamber 2 to a product chamber 3 (not directly shown in this diagram). Dotted line 14 represents a beam of electrons released from the emitter 11. The electron chamber 2 contains atmosphere 15, which may include a low pressure and/or inert gas. FIG. 3 also depicts focusing cup 16 that is used to alter the path or shape or both of the electrons 14. The electric field generated by the focusing cup 16 affects the electrons and begins to organize the electrons into an electron beam, for example.

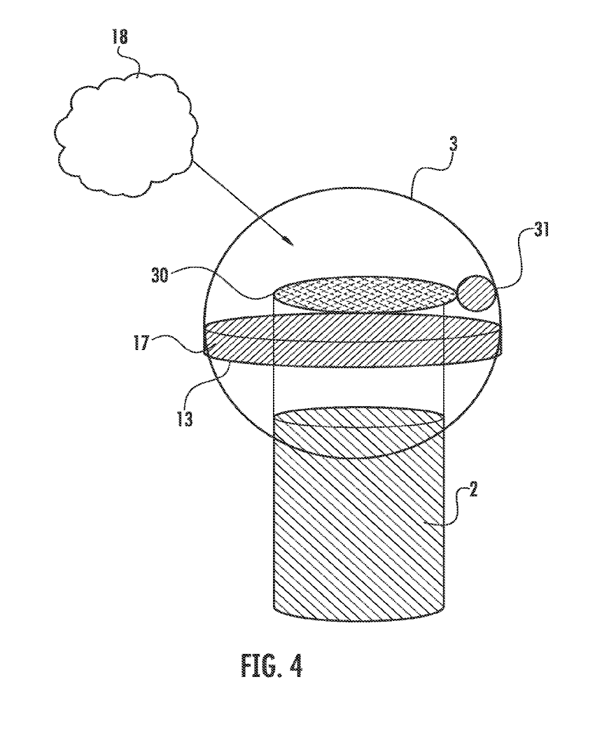

The embodiment of the sterilizer shown in FIG. 4 can optionally be suited, for example, to sterilize a medical instrument or implantable hardware in the operating room. This embodiment can have the advantages of low heat build up and as well as no organic molecule effect on the instrument or implantable device. This embodiment comprises a product chamber 3 coated with a high Z number material (e.g., a material with a Z number of 47 or more, preferably gold) and a grounded open mesh platform 30 for supporting the targeted item during the sterilization process. In addition, shown in FIG. 4 is a vibrating motor 31 and a high volume vacuum pump. The electron chamber 2 connects to the product chamber 3. The product chamber 3 is represented as a ball, but can be an oblong sphere as well. In this type of embodiment the product chamber can have a clamshell configuration where the sphere or oblong sphere can be opened by a hinge mechanism allowing to parts of the sphere to open like a clamshell. An adjusted atmosphere 18 such as a vacuum or an inert atmosphere is shown within the product chamber 3. A mesh platform 30 is shown which holds the targeted item, such as a medical instrument, such as forceps or scalpel. Vibrating coil 31 vibrates the mesh 30 to agitate the targeted item and expose all surfaces to the sterilizing electrons. When product chamber is a clamshell configuration, the coupling connectors 13, 17 of the electron and product chambers 2, 3 can optionally create a seal between the top and bottom portions of the product chamber 3. The seal ensures that the pressure of atmosphere 18 may be reduced or made inert or both and maintained in its adjusted state to facilitate sterilization.

The product chamber 3 and electron chamber 2 of FIG. 4 may optionally share the same atmosphere 18. A valve (not shown) may be employed between electron chamber 2 and product chamber 3 to permit precise control over when or if the atmospheres in the two chambers mix. Alternatively, the electron chamber 2 and product chamber 3 may have permanently separate atmospheres. In such a configuration, the two chambers can be separated, for example, by a non-movable window that permits electrons to pass from the electron chamber 2 to the product chamber 3. The atmospheres in each of the chambers would be independently controlled for pressure and gaseous content.

During a surgical procedure, a surgical instrument or implantable hardware can be rinsed, dried and placed on platform 30 (protocol can differ for different materials or instruments). A vacuum can be drawn on the chamber 3, and can be drawn in about 0.5-10 minutes (or less as described herein) depending on the size of the chambers and the capacity of the vacuum pump employed. Effective 25 keV electrons can be emitted into chamber 3 through aperture 12 while platform 30 is vibrated and/or rotated. At the end of the sterilization cycle, the product chamber 3 can be backfilled with inert gas and the irradiated item is moved to the sterile field or placed in a sterile bag. The inert gas will also aid in cooling the targeted item and reducing oxidation.

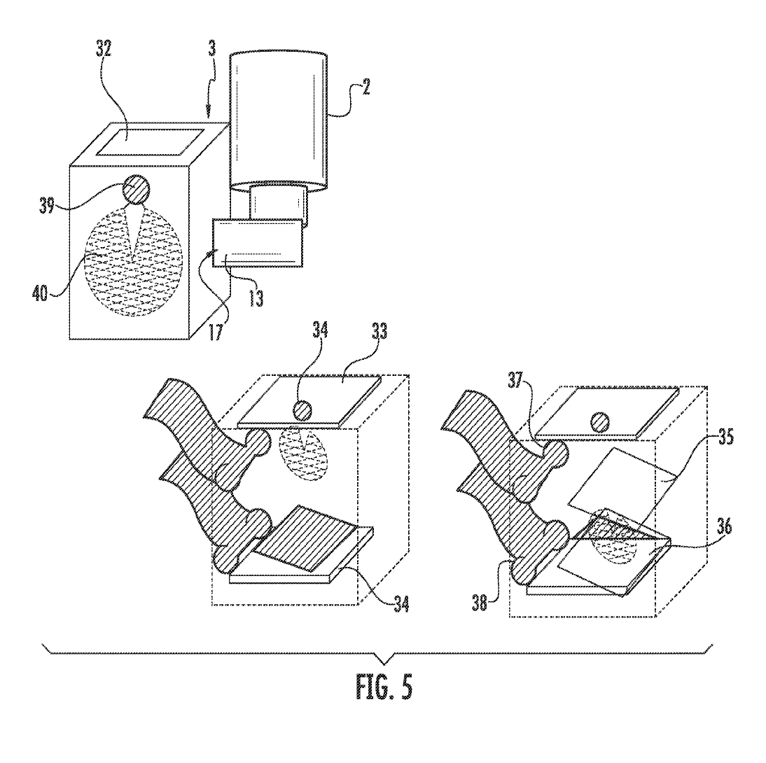

The device shown in FIG. 5 illustrates an embodiment of the sterilizer that is similar to the embodiment of FIG. 4. One difference is that a conductive mesh bag 40 replaces the mesh tray 30 of FIG. 4. Additionally, two entrance rollers 37 and 38, which transport a plastic film into and out of product chamber 3 without breaking the adjusted atmosphere inside product chamber 3. The film can be transported such that it is positioned above and below the instrument to be sterilized so that the upper and lower portions of the film can be heat scaled around the targeted item after it has been sterilized. Before the film is heat sealed around the targeted item, the electron emitter can be used to expose the underside of the upper roll and the top side of the lower roll of film to ensure that the film itself is sterile. After these surfaces of the film have been sterilized, the wire mesh bag 40 is dropped onto one of the plastic films, and then a heat sealing frame top and bottom 33 and 34 which can come down and seal the two pieces of plastic or film, 35 and 36 together. The plastic or film encased instrument can then be disconnected from the roll of film for example, and can be removed from the sterilizer in a sterile manner. The cover 35 can be a heat sensitive cover. Sheet 35 and 36 are shown placed in pressure and heat sensitive holders such that the faces of the covering are faced toward the electrons during the irradiation cycle. Alternatively, a plastic bag open at one end could be placed at the bottom of the chamber. After the sterilization cycle, (including sterilizing of the bag), the bag is drawn over the targeted item and sealed, for example, with a heat seal.

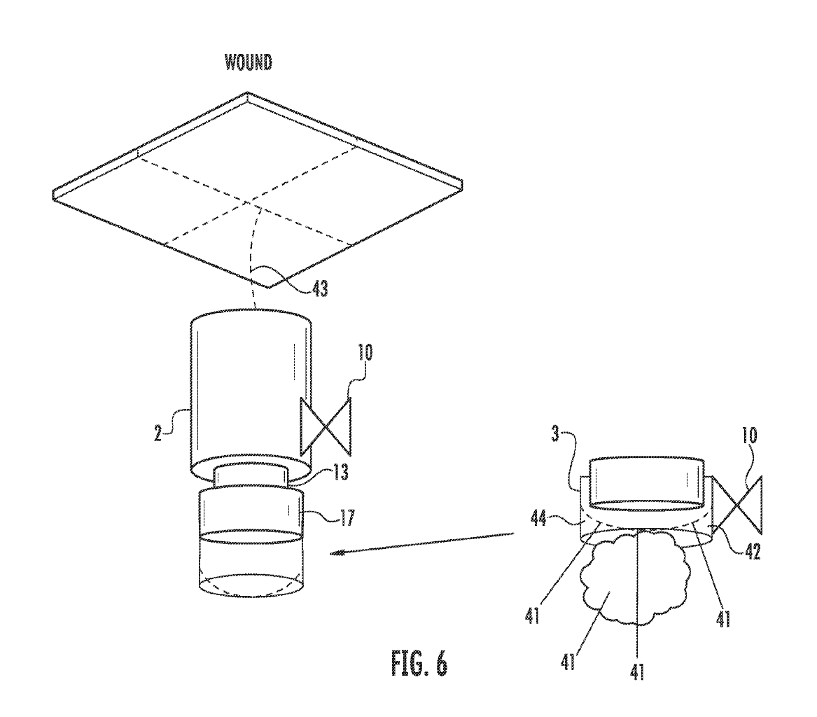

FIG. 6 illustrates an embodiment of the sterilizer that is well suited for sterilizing living tissues such as wounds, burns, and ulcers among others. The electron chamber 2 includes an electron emitter that emits electrons that stream out of the bottom side of electron chamber 2 and through the product chamber 3. Electron chamber 2 is connected to a product chamber 3 through complementary coupling connectors 13 and 17. The product chamber 3 can be an open ended chamber that contains atmosphere 41, which is preferably an inert gas such as helium so that the electrons reach the targeted item with sufficient sterilizing energy. A bottle of helium or inert gas 7 can be attached to valve 10 and connector assembly. As valve 10 is opened, helium or inert gas flows into the chamber 3 at above atmospheric pressure to maintain a positive pressure of gas flowing. This positive pressure displaces air and creates a more favorable localized atmosphere 41 for the electrons in the beam to travel more efficiently. Also shown is a convex (electron permeable window) 42, which is an electron director having a shape attempting to match the exit of the electrons out of the product chamber 3, so that at any given x-y-z coordinate exit point an electron from the convex 42, the electron will travel approximately the same distance through the helium as any other electron leaving from any other x-y-z coordinate of the convex 42. This will have the effect of producing approximately the same amount of energy for each electron at the surface of the wound. The convex 42 can be adapted for different body shapes and different shapes of product chamber 3 and desired helium flow patterns to ensure even exposure of the wound to the electrons in the beam. Acceptable variance from electron to electron is within + or −3%, 5%, 8%, 10%, 20% 50%, or 70%. An indexing surface 43 is shown above the sterilizer to provide a frame of reference for moving the sterilizer in space as it traverses a targeted item with a surface area that is too large to expose at one time. The sterilizer includes systems for sensing its location under the indexing surface 43, moving the sterilizer to other locations under indexing surface 43, and tracking the amount of time spent at each location while the sterilizer is emitting electrons. The sterilizer also includes systems for moving the sterilizer such that the entire surface area of the targeted item receives a dose of electrons that is sufficient to sterilize the targeted item.

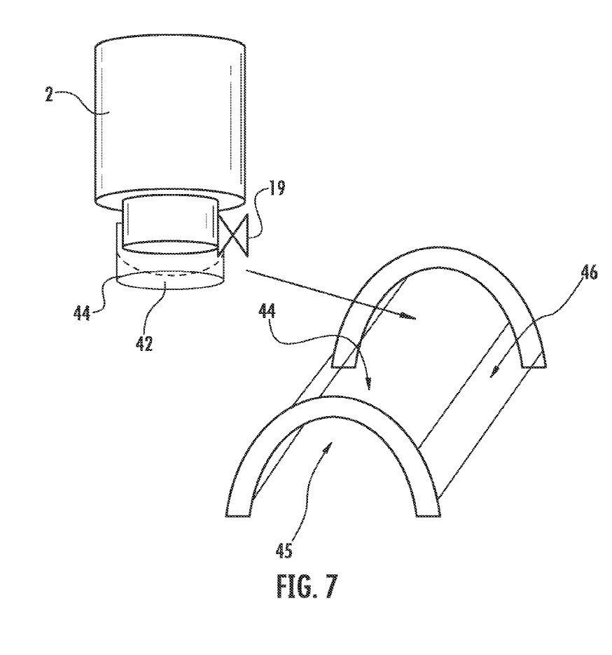

FIG. 7 schematically illustrates another embodiment of a sterilizer for wounds similar to the embodiment of FIG. 6. In FIG. 7, the atmosphere surrounding the wound is controlled via helium a convex (electron permeable window) 42 having an access port that mates with a flexible non-porous drape 45 and a non-permiablizable drape 46. The drapes 45, 46 are flexible enough to conform closely to the wound and will maintain the sterile atmosphere of inert gas to facilitate efficient delivery of the electrons in the beam. Areas of the wound that need not be treated are covered by Vaseline® or some other sterile electron absorbing material.

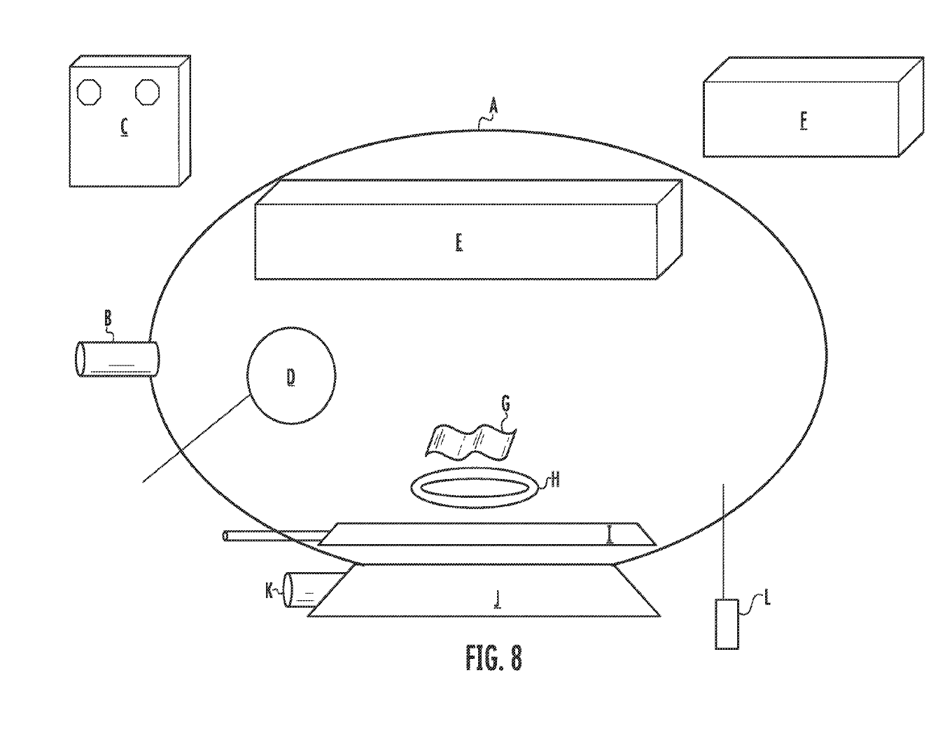

FIG. 8 schematically illustrates a generic embodiment of the sterilizer. FIG. 8 depicts a module having an outer leak proof shell A, which is ovoid in shape. While it is depicted as ovoid, it can be any shape. However, it should be able to withstand a vacuum. Shell A has a valve B to control atmosphere within the shell. Also shown is a human interface module C and energy feedback module D. Power supply E typically provides up to about 25 kV potential with either grounded anode or grounded cathode potentials and current up to about 1 amp. The unit is also shown with an outside power source F. Electron generator G represents any one of a known type of device for generating electrons. More than one electron generator G may be used to alter the exposure pattern of the electrons to the targeted item. The sterilizer may also include an electromagnetic coil H for narrowing or diffusing the beam and/or altering the direction of the beam. Also shown is an adjustable aperture into the housing I to allow electrons to pass from the housing. A connector J is shown for mechanically interfacing with a product chamber, now shown. As described above, the product chamber may or may not share the same atmosphere with the shell A. An optional valve K is connected to connector J to when it is desired to control the atmosphere in the product chamber separately from the atmosphere in the shell A. The sterilizer also includes an electrical connector L to the secondary irradiation device.

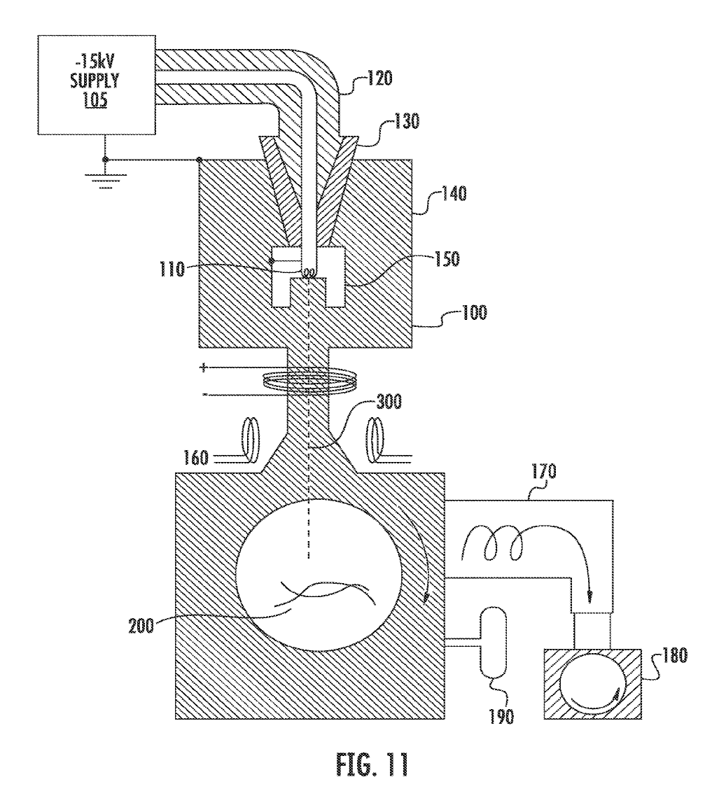

FIG. 11 schematically illustrates in a sectional view an electron emitter that is well suited for use in the sterilizer and that is connected to a product chamber with a rotating holder for the targeted item. The emitter shown in FIG. 11 is a capable of operation in a vacuum greater than 10⁻⁵ Torr and up to a pressure of 10⁻¹ Torr. The emitter 100 uses a filament or a plasma as the source of electrons. For example, a magnetron source such as those used for sputtering, is able to maintain a highly ionized plasma in a relatively low vacuum pressure (a higher absolute pressure) when compared to the pressure that would normally support a plasma. This is due to a magnetic field used to confine electrons to a circular orbit, thus substantially increasing the number of collisions between electrons and the residual gas molecules in the vacuum. Typically, this arrangement is used as an ion source. However for a sterilizer, an appropriately biased acceleration voltage pulls electrons from the plasma to guide them to collide with a targeted item. The energy levels and the number of the electrons that collide with the target are controlled by altering the acceleration voltage, the atmosphere through which the electrons travel, and/or the confinement magnetic field.

The exemplary emitter 100 of FIG. 11 includes power supply 105 that is rated for 15 kV. High voltage cable 120 transmits power from power supply 105 to a coiled iridium filament 110 that is coated with yttria. The filament 110 is contained within a vacuum enclosure 140. It is well known that such an iridium filament can be heated to high temperature without significant chemical reactions with the atmosphere due to its highly stable oxide. However, since iridium has a high work function it is not possible to directly generate significant electrons currents. For this reason, a stable low work function material is applied to the iridium, such as thorium oxide or yttria. Since thorium oxide has a high level of natural radioactivity we have developed a low work function emitter composed of the oxide of yttrium, europium and vanadium. Cerium oxide and yttria are also preferred materials. Electrons are emitted from filament 110 inside focusing cup 150. Electrons are drawn due to the bias from the surrounding grounded chamber and directed toward the targeted item in an electrons 300 using voltage applied to the deflection and focusing coils 160.

Pressure inside the vacuum enclosure 140 is regulated by turbo pump 170 and rough pump 180 using a butterfly valve to control pumping speed and/or a valve to introduce gas in to the chamber, with the pressure being readable via pressure gauge 190. The targeted item is placed in a rotating bowl or tumbler 200 so that the various surfaces of the item are evenly exposed to electrons in the electrons 300. The deflection and focusing coils 160 may also be used to move and focus/defocus the electrons 300, which further ensures that all surfaces of the targeted item are exposed sufficiently to the sterilizing effect of electrons.

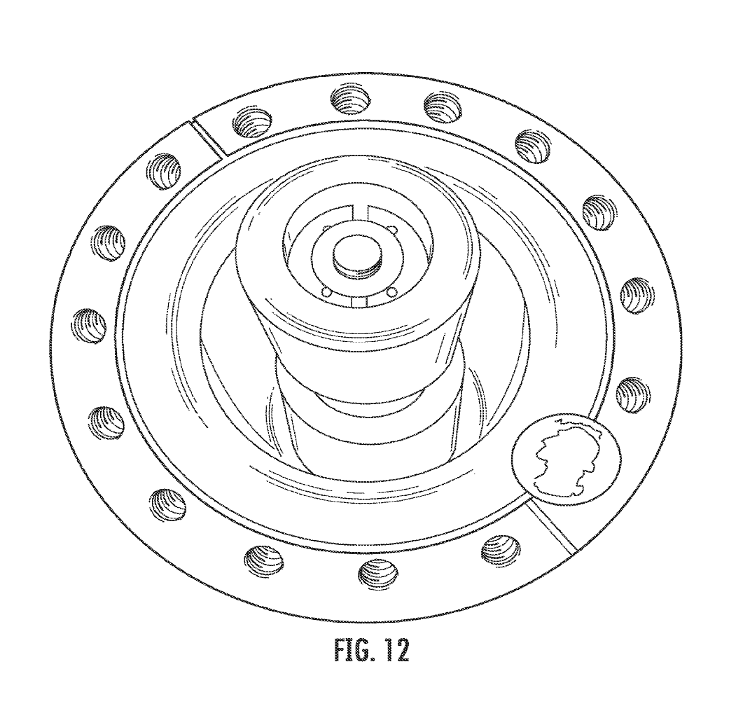

FIG. 12 illustrates a prototype of a plasma-based electron emitter which can be used in the sterilizer. Represented is a portion of a vacuum chamber defined by the flange. The bell shaped component is the focusing cup. Inside the focusing cup, there is a ceramic high voltage receptacle surrounding a magnet or electromagnet. Plasma is generated in the atmosphere around the magnet. The atmosphere may include residual water vapor and atmospheric air inside the chamber that remain as the pressure inside the chamber is lowered. Other gases fed into the chamber (e.g., helium or argon) may support the generation of a plasma.

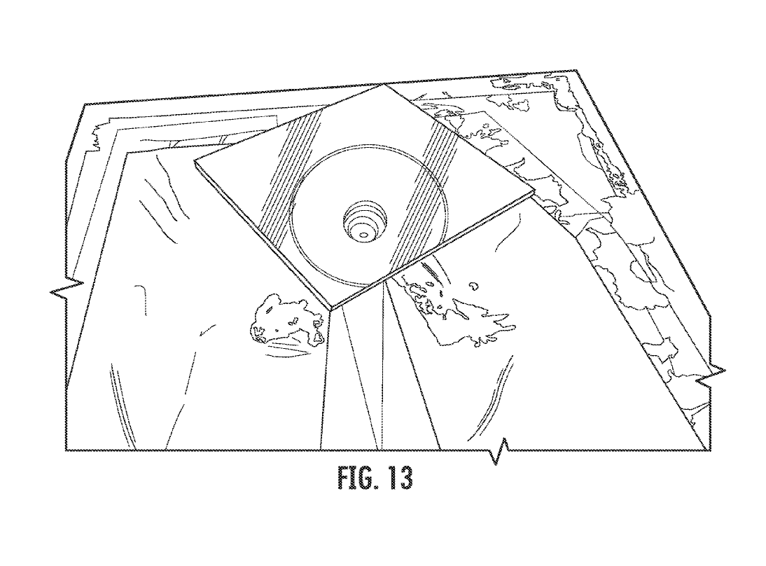

FIG. 13 illustrates a prototype of the plasma-based electron emitter shown in FIG. 12 while the emitter is in operation. A mirror, shown approximately in the center of the figure, is placed on a phosphorous coated screen that is inside a chamber that is free of electrical fields. The mirror reflects the image of the electron source so that the camera can capture an image of the emitter in operation. The plasma generated by the emitter is visible as the bright white ring near the center of the reflected image. The electrons being drawn from the plasma cast the purple glow that is visible just above the ring of plasma. The electrons are ejected from the plasma through a narrow aperture into the chamber which is free of electrical fields. Electrons being emitted by the source cause the phosphorous coated screen to glow green.

### A. DEFINITIONS

1. A, an, the

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. About

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

3. Activity

Activity or like terms refers to the actions or states disclosed herein, such as the actions or states of cell proliferation, modulating binding or modulating a signaling pathway, and transactivation and downstream transactivation.

4. Component

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

5. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc. are treated as in a parallel experiment except for omission of the procedure or agent or variable etc. under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

6. Inhibit

By “inhibit” or other forms of inhibit means to hinder, suppress, or restrain a particular characteristic. It is understood that this is typically in relation to some standard or expected value, in other words it is relative, but that it is not always necessary for the standard or relative value to be refereed to. For example, “inhibits bioburden” means hindering or restraining the amount of bioburden that takes place relative to a standard or a control. It is understood that wherever one of these words is used it is also disclosed that it could be, for example, at least 1%, 5%, 10%, 20%, 50%, 100%, 500%, 1000, 10,000, 100,000, 1,000,000% inhibited from a control.

7. Increase

By “increase” or like terms means raising of an event or characteristic. It is understood that this is typically in relation to some standard or expected value, in other words it is relative, but that it is not always necessary for the standard or relative value to be referred to. For example, “increases bioburden” means raising the amount of bioburden relative to a standard or a control. It is understood that wherever one of these words is used it is also disclosed that it could be, for example, at least 1%, 5%, 10%, 20%, 50%, 100%, 500%, 1000, 10,000, 100,000, 1,000,000% raised from a control.

8. Isolated

As used herein, the terms “isolated,” “purified,” or like terms refer to material which is substantially or essentially free from components that normally accompany it as found in its native state or substantially free from non material components, such as at least 50%, 60%, 70%, 80%, 90%, 95%, 98%, or 99% material.

9. Material

Material is the tangible part of something (chemical, biochemical, biological, or mixed) that goes into the makeup of a physical object.

10. Modulate

To modulate, or forms thereof, means either increasing, decreasing, or maintaining an activity. It is understood that wherever one of these words is used it is also disclosed that it could be. It is understood that wherever one of these words is used it is also disclosed that it could be, for example, at least 1%, 5%, 10%, 20%, 50%, 100%, 500%, 1000, 10,000, 100,000, 1,000,000% modulated from a control.

11. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

12. Optional

“Optional” or “optionally” means that the subsequently described event or circumstance may or may not occur, and that the description includes instances where said event or circumstance occurs and instances where it does not.

13. Prevent

By “prevent” or like terms or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

14. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

15. Reduce or decrease

By “reduce” or “decrease” or other forms of reduce or decrease or like terms means lowering of an event or characteristic. It is understood that this is typically in relation to some standard or expected value, in other words it is relative, but that it is not always necessary for the standard or relative value to be referred to. For example, “reduces gene product” means lowering the amount of gene product that takes place relative to a standard or a control. It is understood that wherever one of these words is used it is also disclosed that it could be, for example, at least 1%, 5%, 10%, 20%, 50%, 100%, 500%, 1000, 10,000, 100,000, 1,000,000% reduced from a control.

16. References

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

17. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

18. System

A system or like terms as used herein refers to an interdependent group of items forming a unified whole. For example, a computer system are the parts, such as a process, a memory storage device, and other parts which can be used to form a functioning computer. Also for example, a system can be made up of parts of a sterilizer. For example, the system can be a sterilizer wherein the system comprises an electron emitter and vacuum pump. In this type of system, for example, the system could be a sterilizer, having an electron emitter and a vacuum pump. Those of skill in the art, given the information herein, can create systems around particular pieces of information, once the information is provided, such as information about an electron emitter or a vacuum pump.

19. Substance

A substance or like terms is any physical object. A material is a substance. Molecules, ligands, markers, cells, proteins, and DNA can be considered substances. A machine or an article would be considered to be made of substances, rather than considered a substance themselves.

20. Compositions with similar functions

It is understood that the machines and components disclosed herein have certain functions, such as emitting electrons. Disclosed herein are certain structural requirements for performing the disclosed functions, and it is understood that there are a variety of structures which can perform the same function which are related to the disclosed structures, and that these structures will ultimately achieve the same result, for example production of electrons.

### B. METHODS OF MAKING THE COMPOSITIONS

The compositions, devises, and articles disclosed herein and the various components and materials and articles necessary to perform the disclosed methods can be made using any method known to those of skill in the art for that particular reagent or compound, or material or article or device, unless otherwise specifically noted.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

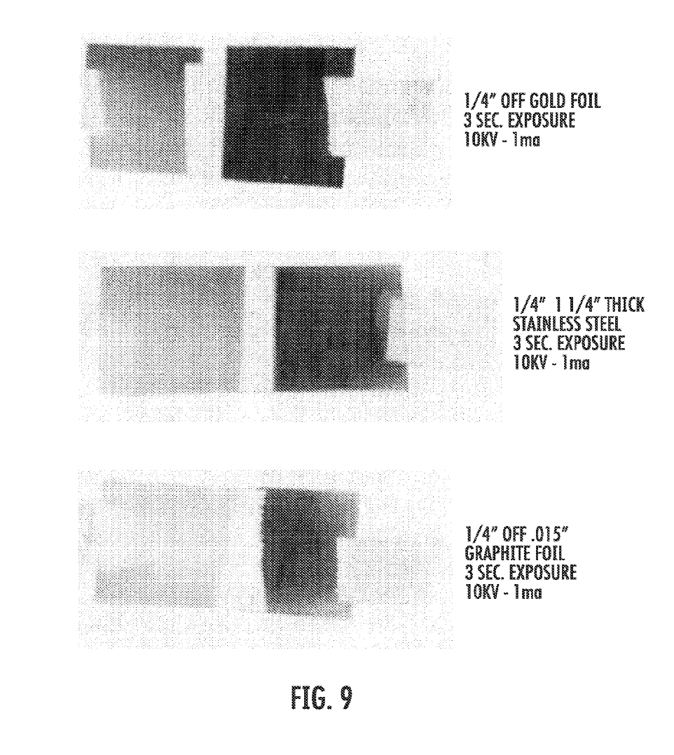

FIG. 9 depicts the results of an experiment to measure the ability of three different potential materials for the lining of the product chamber to reflect electrons with the approximate energies contemplated in the disclosed sterilizer. The three different materials were gold, stainless steel, and graphite. The experiment emitted electrons from the electron emitter, so that they struck one of the three materials. There was a film ¼ inch from the surface of the material, so that electrons that passed through the material also passed the film without hitting it, and only struck the film if on a reflective path. The electrons were emitted for a 3 second pulse at 10 kV and 1 mA. The film therefore measures the extent to which electrons were not absorbed by the material but and rebounded back toward the film. These rebounding electrons, in a sterilization context, would be available for sterilizing whatever they hit. The results of this experiment showed that gold reflected more electrons than stainless steel and stainless steel reflected more electrons than graphite. This indicates that all three of these types of materials, and analogs, would be suitable for use as a material lining the product chamber of an irradiator, and that gold and stainless steel would be preferred electron multipliers relative to graphite.

### 2. Example 2

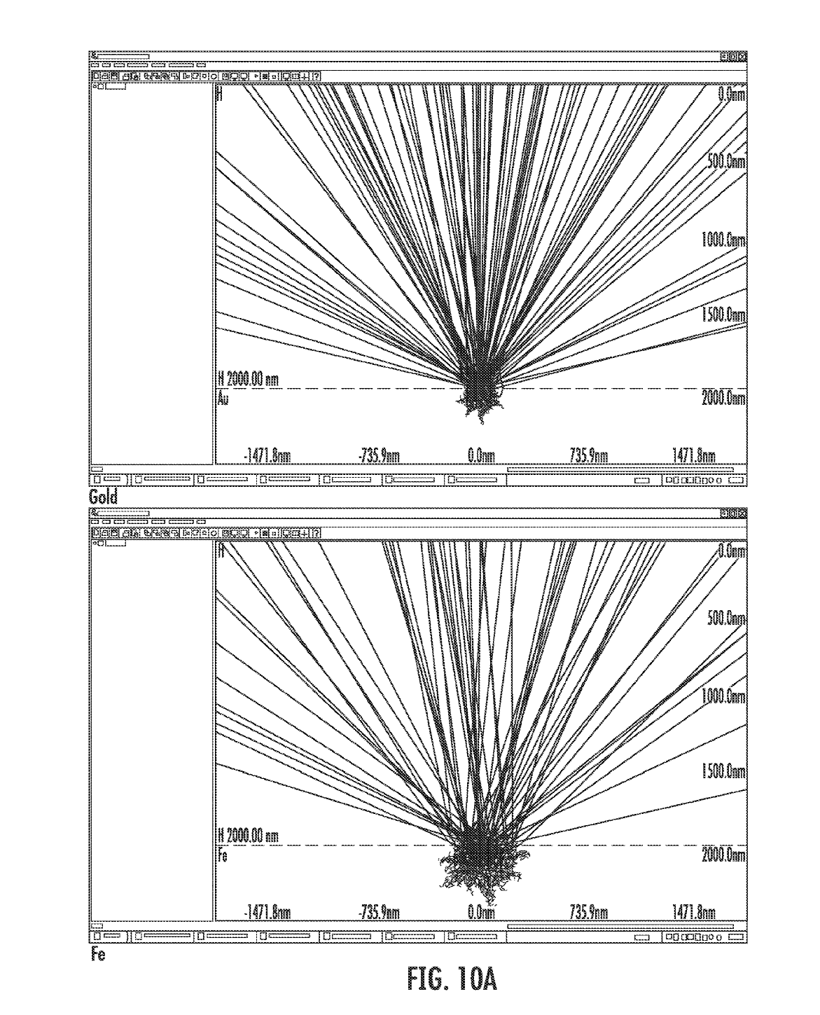

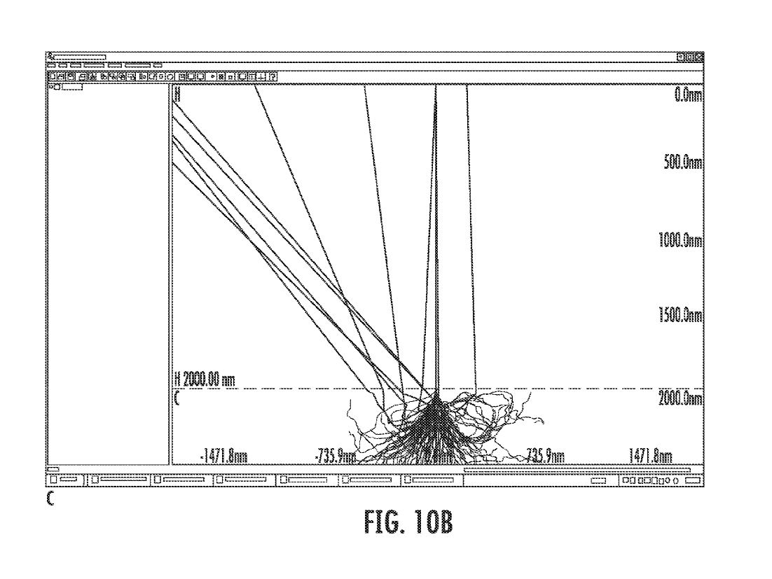

FIGS. 10A and 10B illustrate the results from a simulation of an electron striking a point and reflecting in some direction based on probability. The model is run by continuing to fire additional electrons at the same point and proceeding trough a probability scale. The results are shown for Gold, Fe, and C, and indicate that Gold has the highest dispersive and most efficiency relative to Fe, and Fe, has more dispersive activity and more efficiency than C. Squiggly lines below the impact line indicate that more of the electrons in C are being absorbed than in Fe, than in Gold, because C is less dense than Fe and Fe is less dense than Gold.

### 3. Example 3 Sterilization Examples

### a) Materials and Methods

### (1) The Pathogen

Spores from Bacillus pumilus were used for testing. They came from Spore Suspension 7066921 obtained from MesaLabs. The spores were obtained and kept at 4 degrees C. per the instructions from the manufacturer.

Used the standard por solution from MesaLabs. The Agar used is Tryptic Soy Agar (TSA) (Soybean-Casein Digest Agar). The final pH is 7.3=/−0.2 and the formula is as follows: Pancreatic Digest of Casein—15.0 grams/Liter; Papaic Digest of Soybean—5.0 grams/Liter; Sodium Chloride—5.0 grams/Liter; Agar—15.0 grams/Liter.

### (2) The Method of Inoculating

Sheets of metal, made from an 316 alloy were cut into 6 by 24 mm strips. These strips were then heat sterilized at 375 degrees F. for 1 hour in a metal box.

The test strips were then removed from the box at a clean bench, and 7 droplets of the Bacillus pumilus solution were placed onto one side of the strip. The strips were allowed to air dry on the clean bench.

Sets of four strips were made at a time. Exposed 3 strips to irradiation conditions, the fourth strip was used as a positive control.

### (3) The Method of Irradiating

A beam of electrons was produced from the irradiation apparatus which was approximately 3 mm×3 mm. The beam was a 15 kV at 1 mAmp. This is Watts/0.625 watts/cm(2) XXX. The beam was scanned over an area of 3×8 mm passes covering the entire 6×24 mm strip in 30 seconds, 60 seconds, or 120 s exposure times.

The irradiation was performed at a vacuum of 2×10⁻⁴ tor for all experiments.

After the prescribed time of irradiation, the internal manipulator arm of the irradiation device was used to move the irradiated strip into a sterilized canister, which was covered in the device, before air was let back into the chamber. The covered canister was then removed and the snap seal was covered with taped to maintain the seal of the canister and its top.

### (4) The Method of Assaying

### (a) General Procedure

There are 11 general steps that can be done to determine the number of bacteria in a given sample.

### (i) Step 1.0

Aseptically 10 BI's into a sterile 250 ml blender cup containing 100 ml of chilled, processed water or pool contents of each ampoule into a sterile screw-capped 10 ml test tube (if processing ampoules, skip to step 4.0; if processing metal product, aseptically transfer 1 carrier into a water blank containing 9.9 ml sterile, processed water with 0.1 ml of Tween 80 and lml of 3 mm sterile glass beads. Vortex for 2 minutes.

Insert 10 ml tube into sonicator (38.5-40.5 KHz, full wave industrial stack transducer for 10 minutes.

Vortex again for 2 minutes. Skip to step 4.0)

### (ii) Step 2.0

Blend 3-5 minutes to a homogeneous pulp of component fibers.

### (iii) Step 3.0

Aseptically transfer a 10 ml aliquot from the blender cup into a sterile, screw-capped 10 ml test tube. Label each tube with Lot #, Temperature and Length of Exposure.

### (iv) Step 4.0

Heat shock tubes in a water bath (10 minutes at 80°-85° C. for mesophiles, 15 minutes at 95°-100° C. for thermophiles.) Immediately cool tubes in a water bath of 0°-4° C. Do not heat shock BIs exposed to sterilant within 2 hours of exposure.

### (v) Step—5.0

Vortex the tubes for 15-20 seconds.

### (vi) Step—6.0

Perform serial dilutions by pipetting out 1.0 ml of the aliquot into another sterile, screw-capped 10 ml test tube containing 9.0 ml of sterile, processed water. Repeat from step 5 until desired dilution factor is reached.

### (vii) Step—7.0

At the dilution factor expected to yield 30-300 CFU, pipette out 1.0 ml into each of three Petri plates.

Repeat for final 2 dilution.

### (viii) Step—8.0

Within 20 minutes, add to each plate approximately 20 ml of TSA, pre-sterilized and cooled to 47°+2° C.

Swirl to distribute spores evenly in agar and allow to solidify.

### (ix) Step 9.0

Invert and incubate the plates (30°-35° C. for mesophiles, 55°-60° C. for thermophiles or as otherwise recommended by the manufacturer)

### (x) Step 10.0

Examine all plates at 24 hrs and record results after 48 hrs. Record on the back the number of colony forming units (CFU's) per plate. Record the average following page.

### (xi) Step 11.0

Calculate the average number of CFU's per carrier from the above data using the formulas on the following page.

### (b) Thermopilus Bacillus Tests

A population assay was performed on each metal strip.

Each strip was aseptically transferred 1 metal strip into a water blank containing 9.9 ml of sterile, processed water with 0.1 ml of Tween 80 and 1 ml of 3 mm sterile glass beads. Vortex for 2 minutes. Insert 10 ml tube into sonicator (38.5-40.5˜KHz full wave industrial stack transducer) for 10 minutes. Vortex again for 2 minutes. Skip to step 4.0).

The tubes were heat shocked in a water bath for 10 minutes at 80 degrees C. The tubes were immediately cooled in a water bath of 0 degrees to 4 degrees C.

The tubes were then vortexed for 15-20 seconds.

Serial dilutions were performed by pipetting out 1.0 ml of the aliquot into another screw-capped 10 ml test tube containing 9.0 ml of sterile, processed water. The tubes then under went serial dilution by repeated vortexing and 10 fold dilution with sterile, processed water.

At a dilution expected to yield 30-300 CFUs (colony forming units), 1 ml was pipetted in each of three Petri plates.

Within 20 minutes, 20 ml of TSA (pre-sterilized and cooled to 49 degrees C.) was added to each plate.

The plates were inverted and incubated at 30-35 degrees C. The plates were examined at 24 hours and 48 hours. All plates were recorded and the average number of CFUs were obtained.

### b) Results

After performing the assay for determining CFUs, it was determined that for a 30 second exposure, 5×100 CFUs were formed, for 120 seconds 0 CFUs were formed, and for 360 seconds 0 CFUs were formed. The positive control produced 2.4×106 CFUs.

1. A sterilizer, comprising:
a low energy electron emitter; and
a product chamber, wherein the low energy electron emitter is configured to emit one or more electrons having an energy less than or equal to 25 keV into the product chamber.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. The sterilizer of claim 1, wherein the product chamber is maintained at a vacuum sufficiently low to prevent the one or more electrons from producing a plasma.

14. The sterilizer of claim 1, wherein the product chamber is maintained at a vacuum less than or equal to 10⁻² Torr.

15. (canceled)

16. The sterilizer of claim 13, wherein the low energy electron emitter and the product chamber are under a same atmosphere.

17. The sterilizer of claim 16, further comprising an electron chamber configured to house the low energy electron emitter.

18. The sterilizer of claim 17, wherein the electron chamber and the product chamber are in fluid communication.

19. The sterilizer of claim 13, further comprising a vacuum pump operatively connected to the product chamber, the vacuum pump being configured to generate and maintain the vacuum.

20. (canceled)

21. (canceled)

22. (canceled)

23. (canceled)

24. (canceled)

25. The sterilizer of claim 1, further comprising a high voltage generator operably coupled to the low energy electron emitter.

26. The sterilizer of claim 25, wherein the high voltage generator is configured to generate at least a voltage of between 5 kV and 25 kV.

27. The sterilizer of claim 1, wherein the low energy electron emitter is at least one of a filament, a cold cathode, or a dispenser cathode, an oxide coated cathode, a photocathode, a thermionic emitter, an electron multiplier and a plasma.

28. The sterilizer of claim 1, wherein at least a portion of the product chamber is coated with a high Z number material.

29. The sterilizer of claim 1, wherein at least one of the electrons is backscattered at least one time in the product chamber.

30.-178. (canceled)
